(19) United States
(12) Patent Application Publication
(10) Pub. No.: US 2022/0139029 A1
(43) Pub. Date: May 5, 2022

(54) SYSTEM AND METHOD FOR ANNOTATION OF ANATOMICAL TREE STRUCTURES IN 3D IMAGES

(71) Applicant: Covidien LP, Mansfield, MA (US)

(72) Inventors: Ariel Birenbaum, Raanana (IL); Ofer Barasofsky, Tel Mond (IL)

(73) Assignee: Covidien LP, Mansfield, MA (US)

(21) Appl. No.: 17/518,421

(22) Filed: Nov. 3, 2021

Related U.S. Application Data
(60) Provisional application No. 63/166,114, filed on Mar. 25, 2021.
(60) Provisional application No. 63/110,271, filed on Nov. 5, 2020.

(51) Int. Cl. G06T 17/00; G06F 3/0485; G06F 3/0481; G06F 3/0484; G06T 19/20; G16H 50/50; G16H 30/40

(52) U.S. Cl. CPC G06T 17/00; G06F 3/0485; G06F 3/04815; G06F 3/04845; G06T 19/20; G16H 50/50; G06T 2219/004; G06T 2210/41; G06T 2200/24; G06F 2203/04806; G06F 3/04842; G06T 2219/2016; G16H 30/40

(57) ABSTRACT

Systems and method of manually and automatically generating 3D models and correcting 3D models by identifying vasculature withing image scan data and constructing a series of connected segments, each segment of the 3D model having a diameter of an oblique view of the vasculature associated with that segment.

### FIELD

This disclosure is directed to systems and method of annotating anatomical tree structures in 3D images. In particular, the disclosure is directed to a software application configured to generate three-dimensional models from computed tomography and other image type data sets.

### BACKGROUND

During a surgical procedure, clinicians often use CT images for determining a plan or pathway for navigating through the luminal network of a patient. Absent software solutions, it is often difficult for the clinician to effectively plan a pathway based on CT images alone. This challenge in creating paths to certain targets is especially true in the smaller branches of the bronchial tree where CT images typically do not provide sufficient resolution for accurate navigation.

While the software solutions for pathway planning a pathway through the luminal networks of, for example the lungs, are great for their intended purpose, they do not assist the clinicians in planning for thoracic surgeries. Thoracic surgeries are typically performed laparoscopically or via open surgery through the patient's chest. Lobectomies are one such thoracic procedure and is one where an entire lung lobe is removed. One reason for performing a lobectomy is that the lobes of the lung are readily discernable and separated from one another via a fissure. As a result, the vasculature of the lobe is also relatively distinct and can be planned for and can be addressed during the surgery with reasonable certainty. However, in many instances a lobectomy removes too much tissue, particularly healthy lung tissue. This can be critical in determining whether a patient is even a candidate for surgery.

Each lung lobe is composed of either three or four lung segments. These segments are generally independently vascularized. This means that if the individual segments can be identified, and the vasculature related to the segments distinguished from other lobes, a segmentectomy may be undertaken. A segmentectomy procedure can increase the number of patients that are surgical candidates because it enables the surgeon to remove the diseased tissue while leaving all other tissue. The problem with segmentectomy procedures is that while they are more tissue efficient, determining the locations of the relevant vascular structures can be very challenging even for highly trained professionals.

The instant disclosure is directed to addressing the shortcomings of current imaging and planning systems.

### SUMMARY

One aspect of the disclosure is directed to a system for generating a generating a three-dimensional (3D) model of vasculature of a patient a memory in communication with a processor and a display, the memory storing instructions that when executed by the processor: cause a display to display a plurality of images from an image data set in a user interface, the images including at least an axial, sagittal, and coronal view; receive instructions to scroll through at least one of the axial, sagittal, and coronal images; receive an indication of a position of one of the axial, sagittal, and coronal images being within a first portion of a vasculature; snap the remaining images to the position of the received indication; display crosshairs on the images at the position of the received indication; depict the position as a first point in a three-dimensional (3D) view; receive inputs to adjust a level of zoom or a location of the crosshairs in the images; receive an indication that all three crosshairs are located in the center of the first portion of the vasculature; depict a second point in a 3D view at the location of all three crosshairs; depict the first point in an oblique view of the image data set; depict a circle in the oblique view around the first point; receive an input to size the size of the circle to match a diameter of the first portion of the vasculature at the second point; receive an input to add a segment; and display the segment in the 3Dview, where the segment extends from the first point to a first node at the location of the second point. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system where a depiction of the segment is also presented in the axial, sagittal, and coronal images. The system where when further segments of the first portion of the vasculature remain unmodeled, the processor executes instructions to: receive an input to scroll through the images in at least one of the axial, sagittal, and coronal images; receive an input identifying a third point in the first portion of the vasculature in at least one of the axial, sagittal, and coronal images; depict a circle in the oblique view around the second point; receive an input to size the size of the circle to match a diameter of the first portion of the vasculature; receive an input to add a segment; and display the segment in the 3D view, where the segment extends from the first node to a second node at the location of the third point. The instructions are executed in a repeating fashion until the entirety of the first portion of the vasculature is modeled. Following modeling of all the segments of the first portion of the vasculature, the processor executes instructions to: receive instructions to scroll through at least one of the axial, sagittal, and coronal images; receive an indication of a portion of one of the axial, sagittal, and coronal images being within a second portion of the vasculature; snap the remaining images to the position of the received indication; display crosshairs on the images at the position of the received indication; depict the position as a first point in the 3D view; receive inputs to adjust a level of zoom or a location of the crosshairs in the images; and receive an indication that all three crosshairs are located in the center of the vasculature. The segment extends from the first point to a first node at the location of the second point. The first portion of the vasculature are arteries and the second portion of the vasculature are veins. The processor executes instructions to export a 3D model formed of a plurality of the segments to an application for planning a thoracic surgery. The system further including identifying an error in at least one segment of a 3D model formed of a plurality of the segments and inserting a segment before the segment with the error. Following identification of a node is defined between the nodes of the segment containing the error. A diameter of the inserted segment is defined in the oblique view. The segment has a diameter matching the size of the circle around the first point. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A second aspect of the disclosure is directed to a system for correcting a 3D model of vasculature of a patient a memory in communication with a processor and a display, the memory storing instructions that when executed by the processor: select a 3D model for presentation on a display; present the 3D model and axial, coronal and sagittal images from which the 3D model is derived on a user interface; receive an input to scroll or zoom one or more of the images, or receive a selection of a segment of the 3D model; receive an indication of a point in a first segment in the 3D model in need of correction; depict the point in an oblique view of the images; depict a circle in the oblique view around the first point; receive an input to size the size of the circle to match a diameter of the vasculature in the oblique view; receive an input to add a segment; and display the added segment in the 3D model, where the added segment extends from a point defining a beginning of the first segment to the first point in and corrects an error in the 3D model. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system where the processor executes the instructions until the entirety of the 3D model is reviewed and corrected. The system where segments of the 3D model depict arterial vasculature in a first color and venous vasculature in a second color. The processor further executes an instruction to export the correct 3D model to a thoracic surgery planning application. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Yet a further aspect of the disclosure is directed to a method of generating a 3D model of a vasculature of lungs. The method includes displaying a plurality of images from an image data set in a user interface, the images including at least an axial, sagittal, and coronal view; receiving instructions to scroll through at least one of the axial, sagittal, and coronal images; receiving an indication of a position of one of the axial, sagittal, and coronal images being within a first portion of a vasculature; displaying crosshairs on the axial, coronal and sagittal images at the position of the received indication; depicting the position as a first point in a three-dimensional (3D) view; receiving an input to adjust a level of zoom or a location of the crosshairs in the images; receiving an indication that all three crosshairs are located in the center of the first portion of the vasculature; depicting a second point in a 3D view at the location of all three crosshairs; depicting the first point in an oblique view of the image data set; depicting a circle in the oblique view around the first point; receiving an input to size the size of the circle to match a diameter of the first portion of the vasculature around the first point; receiving an input to add a segment; and displaying the segment in the 3D view, where the segment extends from the first point to a first node at the location of the second point. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method where a depiction of the segment is also presented in the axial, sagittal, and coronal images. The method where the segment has a diameter matching the size of the circle around the first point. Where further segments of the first portion of the vasculature remain unmodeled: receiving an input to scroll through the images in at least one of the axial, sagittal, and coronal images; receiving an input identifying a third point in the first portion of the vasculature in at least one of the axial, sagittal, and coronal images; depicting a circle in the oblique view around the second point; receiving an input to size the size of the circle to match a diameter of the first portion of the vasculature; receiving an input to add a segment; displaying the segment in the segmentation view, where the segment extends from the first node to a second node at the location of the third point. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Objects and features of the presently disclosed system and method will become apparent to those of ordinary skill in the art when descriptions of various embodiments thereof are read with reference to the accompanying drawings, of which:

FIG. 1A is a schematic view of human lungs separated by lobes and segments;

FIG. 1B is a schematic view of human lungs separated into segments;

FIG. 2 is a user interface for a thoracic surgery planning platform in accordance with the disclosure;

FIG. 3 is a user interface for a thoracic surgery planning platform in accordance with the disclosure;

FIG. 4 is a user interface for a thoracic surgery planning platform in accordance with the disclosure;

FIG. 5 is a user interface for a thoracic surgery planning platform in accordance with the disclosure;

FIG. 6 is a user interface for a thoracic surgery planning platform in accordance with the disclosure;

FIG. 7 is a schematic view of a workstation in accordance with the disclosure;

FIG. 8 is a user interface of a 3D model generating application in accordance with the disclosure;

FIG. 9A is a flow diagram for generating a 3D model in accordance with the disclosure;

FIG. 9B is a continuation of the flow diagram of FIG. 9A for generating a 3D model in accordance with the disclosure;

FIG. 10 is a user interface of a 3D model generating application in accordance with the disclosure;

FIG. 11 is a user interface of a 3D model generating application in accordance with the disclosure;

FIG. 12 is a user interface of a 3D model generating application in accordance with the disclosure;

FIG. 13 is a user interface of a 3D model generating application in accordance with the disclosure;

FIG. 14 is a user interface of a 3D model generating application in accordance with the disclosure;

FIG. 15 is a user interface of a 3D model generating application in accordance with the disclosure;

FIG. 16 is a user interface of a 3D model generating application in accordance with the disclosure;

FIG. 17 is a user interface of a 3D model generating application in accordance with the disclosure;

FIG. 18 is a user interface of a 3D model generating application in accordance with the disclosure;

FIG. 19 is a user interface of a 3D model generating application in accordance with the disclosure;

FIG. 20 is a user interface of a 3D model generating application in accordance with the disclosure;

FIG. 21 is a user interface of a 3D model generating application in accordance with the disclosure;

FIG. 22 is a user interface of a 3D model generating application in accordance with the disclosure;

FIG. 23 is a user interface of a 3D model generating application in accordance with the disclosure;

FIG. 24 is a user interface of a 3D model generating application in accordance with the disclosure;

FIG. 25 is a flow diagram for correcting a 3D model in accordance with the disclosure.

FIG. 26 is a flow diagram for automatically extending or generating a 3D model in accordance with the disclosure;

FIG. 27 is a schematic view of extending a blood vessel in a 3D model according to the method of FIG. 26;

FIG. 28 is a schematic view of extending a second blood vessel in a 3D model according to the method of FIG. 26;

FIG. 29 is a schematic view of the extending blood vessels in a 3D model according to the method of FIG. 26;

FIG. 30 is a user interface of a 3D model extending or generation application in accordance with the disclosure;

FIG. 31 is a user interface of a 3D model extending or generation application in accordance with the disclosure;

FIG. 32 is a user interface of a 3D model extending or generation application in accordance with the disclosure;

FIG. 33 is a user interface of a 3D model extending or generation application in accordance with the disclosure;

FIG. 34 is a user interface of a 3D model extending or generation application in accordance with the disclosure;

FIG. 35 is a user interface of a 3D model extending or generation application in accordance with the disclosure;

FIG. 36 is a user interface of a 3D model extending or generation application in accordance with the disclosure; and

FIG. 37 is a user interface of a 3D model extending or generation application in accordance with the disclosure;

### DETAILED DESCRIPTION

This disclosure is directed to a system and method of receiving image data and generating 3D models from the image data. In one example, the image data is CT image data, though other forms of image data such as Magnetic Resonance Imaging (MM), fluoroscopy, ultrasound, and others may be employed without departure from the instant disclosure.

In one aspect of the disclosure, a user navigates to the portion of the image data set such that the patient's heart is in view. This allows the user to identify important vascular features around the heart, such as the right and left pulmonary arteries, left and right pulmonary veins, the aorta, descending aorta, inferior and superior vena cava. These larger vascular features are generally quite distinct and relatively uniform in location from patient to patient. These methods and the 3D models generated may be used for a variety of purposes, including for importation into a thoracic surgery planning system, as outlined below.

In a further aspect the disclosure is directed to an annotation method allowing for manually tracing pulmonary blood vessels from mediastinum towards periphery. The manual program described herein can be used for a number of purposes including generating 3D models, performing peer review, algorithms training, algorithms evaluation, and usability sessions as well as allowing for user correction and verification of algorithm-based 3D model generation from CT image data sets.

The tool enables manual annotation of anatomical trees. Separate trees may be generated for each blood vessel that enters/exits the heart. Each tree model is decomposed into a set of cylinder-shaped segments. In one aspect the user marks segment start and end points. An oblique view is displayed, where radius is marked accurately. The segment's cylinder is then added to tree and displayed.

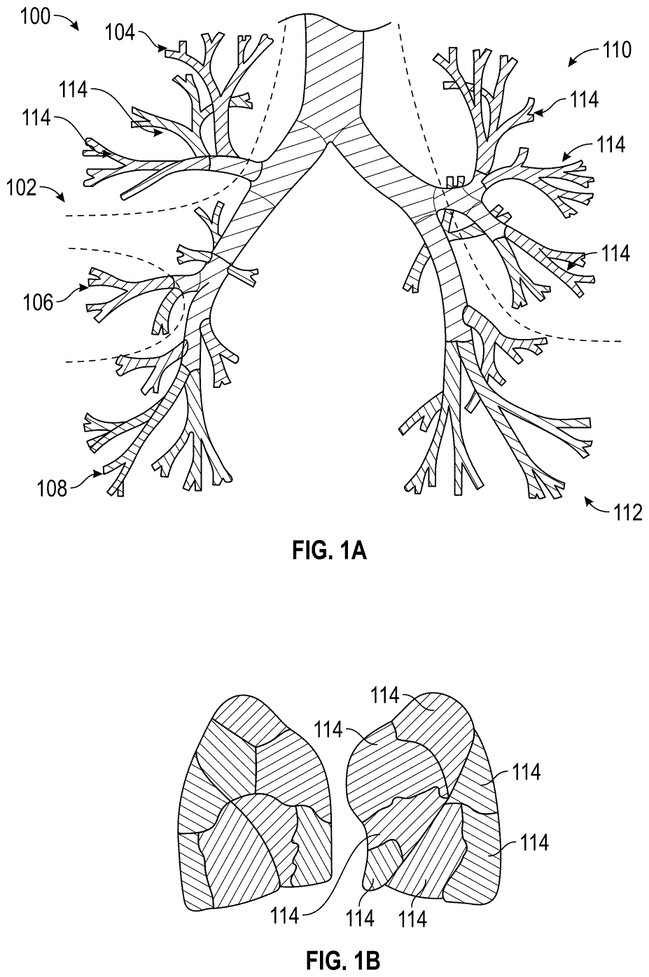

FIG. 1A depicts a schematic diagram of the airways of the lungs 100. As can be seen right lung 102 is composed of three segments, the superior lobe 104, middle lobe 106, and inferior lobe 108. The left lung is comprised of the superior lobe 110 and the inferior lobe 112. Each lobe 104-112 is composed of three or four segments 114, each segment 114 includes a variety of distinct airways 116. FIG. 1B depicts the right and left lungs and with each of the segments 114 as they generally appear in the human body.

As is known to those of skill in the art, the vasculature of the lungs generally follows the airways until reaching the periphery of the lungs where the blood-air barrier (alveolar-capillary barrier) where gas exchange occurs allowing carbon dioxide to be eliminated from the blood stream and oxygen to enter the blood stream as part of normal respiration. However, while the vasculature generally follows the airways, it there are instances where portions of the same blood vessel supplies two or more segments. Particularly the more central vasculature can be expected to supply multiple segments.

Further, in instances where the segmentectomy is the result of a tumor, the tumor, which is a very blood rich tissue, is fed by multiple blood vessels. These blood vessels may in fact be supplying blood to the tumor from different segments of the lungs. As a result, it is critical that the thoracic surgeon be able to identify all of the blood vessels entering the tumor and ensure that they are either sutured closed prior to resection or that a surgical stapler is employed to ensure limit the possibility of the surgeon experiencing an unexpected bleeding blood vessel during the procedure.

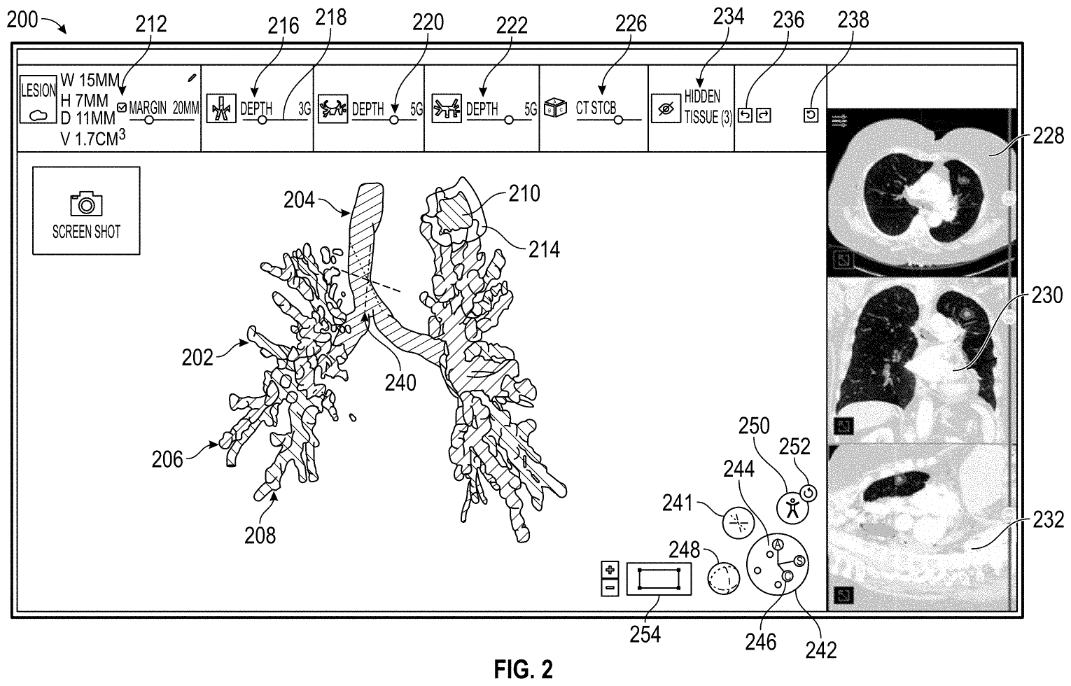

FIG. 2 depicts a user interface 200 of a thoracic surgery planning system. The surgical planning system includes a software application stored in a memory that when executed by a processor performs a variety of steps as described hereinbelow to generate the outputs displayed in the user interface 200. As depicted in the center of the user interface 200 one of the first steps of the software is to generate a 3D model 202. The 3D model 202 is of the airways and the vasculature around the airways is generated from a CT image dataset acquired of the patient's lungs. Using segmentation techniques, the 3D model 202 is defined from the CT image data set and depicts the airways 204 in one color, the veins 206 in a second color, and the arteries 208 in a third color to assist the surgeon in distinguishing the portions of the anatomy based on color.

The application generating the 3D model 202 may include a CT image viewer (not shown) enabling a user to view the CT images (e.g., 2D slice images from the CT image data) prior to generation of the 3D model 202. By viewing the CT images the clinician or other user may utilize their knowledge of the human anatomy to identify one or more tumors in the patient. The clinician may mark the position of this tumor or suspected tumor in the CT images. If the tumor is identified in for example an axial slice CT image, that location may also be displayed in for example sagittal and coronal views. The user may then adjust the identification of edges of the tumor in all three views to ensure that the entire tumor is identified. As will be appreciated, other views may be viewed to assist in this process without departing from the scope of the disclosure. The application utilizes this indication of location provided by the clinician to generate and display an indicator of the location of the tumor 210 in the 3D model 202. In addition to manual marking of the location of the tumor, there are a variety of known automatic tumor identification tools that are configured to automatically process the CT image scan and to identify the suspected tumors.

The user interface 200 includes a variety of features that enable the clinician to better understand the physiology of the patient and to either enhance or reduce the volume of information presented such that the clinician is better able to understand. A first tool is the tumor tool 212 which provides information regarding the tumor or lesion that was identified in the 2D CT image slices, described above. The tumor tool 212 provides information regarding the tumor such as its dimensions. Further, the tumor tool 212 allows for creation of a margin 214 around the tumor 210 at a desired distance from edges of the tumor 210. The margin 214 identifies that portion of healthy tissue that should be removed to ensure that all of the cancerous or otherwise diseased tissue is removed to prevent future tumor growth. In addition, by providing an indicator of the margin 214, the user may manipulate the 3D model 202 to understand the vasculature which intersects the tumor 210. Since tumors are blood rich tissue there are often multiple blood vessels which lead to or from the tumor. Each one of these needs to be identified and addressed during the segmentectomy procedure to ensure complete closure of the blood vessels serving the tumor. Additionally, the margin may be adjusted to or changed to limit the impact of the procedure on adjacent tissue that may be supplied by common blood vessels. For example, the margin is reduced to ensure that only one branch of a blood vessel is transected and sealed, while the main vessel is left intact so that it can continue to feed other non-tumorous tissue. The identification of these blood vessels in an important feature of the disclosure.

The next tool depicted in FIG. 2 is an airway generation tool 216. The airway generation tool 216 allows the user to determine how many generations of the airways are depicted in the 3D model 202. As will be appreciated, image processing techniques have developed to allow for the identification of the airways throughout the lung tissue. There are up to about 23 generations of airway in the lungs of a human from the trachea to the alveolar sacs. However, while very detailed 3D models can be generated, this detail only adds to the clutter of the 3D model and renders the model less useful to the user as the structures of these multiple generations obscures structures. Thus, the airway generation tool 216 allows the user to limit the depicted generations of the airways to a desired level that provides sufficient detail for the planning of a given procedure. In FIG. 2 the airway generation tool 216 is set to the third generation, and a slider 218 allows for the user to alter the selection as desired.

Both a venous blood vessel generation tool 220 and an arterial blood vessel generation tool 222 are depicted in FIG. 2. As with the airway generation tool 216, the venous blood vessel generation tool 220 and the arterial blood vessel generation tool 222 allow the user to select the level of generations of veins and arteries to depict in the 3D model. Again, by selecting the appropriate level of generation the 3D model 202 may be appropriately decluttered to provide useable information to the user.

While these blood vessel generation tools 220 and 222 and the airway generation tool 216 are described here as being a global number of generations of blood vessels and airways displayed in the 3D model 202, they may also be employed to depict the number of generations distal to a given location or in an identified segment of the 3D model 202. In this manner the clinician can identify a particular branch of an airway or blood vessel and have the 3D model 202 updated to show a certain number of generations beyond an identified point in that airway or blood vessel.

In accordance with the disclosure, a generation algorithm has been developed to further assist in providing useful and clear information to the clinician when viewing 3D models having airways and blood vessels both displayed in the UI 200. Traditionally in a luminal network mapping each bifurcation is treated as creation of a new generation of the luminal network. The result is that a 3D model 202 may have up to 23 generations of, for example, the airways to the alveolar sacs. However, in accordance with one aspect of the disclosure a generation is defined differently by the software application generating the 3D model. The application employs a two-step model. The first step identifies the bifurcation in a luminal network. In a second step, at the bifurcation both subsequent branching lumens are measured and if one of the branching lumens has a diameter that is similar in size to the lumen leading to the bifurcation, that branching lumen segment is considered the same generation as the preceding segment. As an example, a branching lumen of “similar size” is one that is at least 50% of the size of the lumen leading to the bifurcation. The result is that a clearer indication of the luminal network from the root lumen is depicted in the 3D model at lower levels of generation. Again, this eliminates much of the clutter in the 3D model providing better actionable data for the clinician.

Additional features of the user interface 200 include a CT slice viewer 226. When selected, as shown in FIG. 2, three CT slice images 228, 230, and 232 are depicted in a side bar of the user interface 200. Each of these CT slice images includes its own slider allowing the user to move alter the image displayed along one of three axes (e.g., axial, coronal, and sagittal) of the patient to view portions of the patient's anatomy. The features identified in the 3D model 202 including airways, venous blood vessels, and arterial blood vessels are also depicted in the CT slice images to provide for greater context in viewing the images. The CT slice images may also be synchronized with the 3D model 202, allowing the user to click on any point in the 3D model 202 and see where that point is located on the CT views. This point will actually be centered in each of the CT slice images 228, 230, and 232. Further, this synchronization allows the user to click on any branch in the 3D model 202 and see where that branch is located on the CT slice images 228, 230, 232. An expand icon 233 in the lower left-hand corner of each CT slice image 228, 230, 232 allows the CT slice image to replace the 3D model 202 in the main display area of the user interface 200.

A hidden tissue feature 234 allows for tissue that is hidden from the viewer in the current view of the 3D model 202 to be displayed in a ghosted or outlined form. Further, toggles 236, and 238 allow for the 3D model 202 to be flipped or rotated.

As described herein there are a variety of tools that are enabled via the UI 200. These tools may be in the form of individual buttons that appear on the UI 200, in a banner associated with the UI 200, or as part of a menu that may appear in the UI 200 when right or left clicking the UI 200 or the 3D model 202. Each of these tools or the buttons associated with this is selectable by a user employing the pointing device to launch features of the application described herein.

Additional features of the user interface 200 include an orientation compass 240. The orientation compass provides for an indication of the orientation of the three primary axes (axial, sagittal, and coronal) with respect to the 3D model. As shown the axes are defined as axial in green, sagittal in red, and coronal in blue. An anchoring tool 241 when selected by the user ties the pointing tool (e.g., mouse or finger on touch screen) to the orientation compass 240. The user then may use a mouse or other pointing tool move the orientation compass 240 to a new location in the 3D model and anchor the 3D model 202 in this location. Upon release of the pointing tool, the new anchor point is established and all future commands to manipulate the 3D model 202 will be centered on this new anchor point. The user may then to drag one of the axes of the orientation compass 240 to alter the display of the 3D models 202 in accordance with the change in orientation of the axis selected.

A related axial tool 242 is can also be used for to change the depicted orientation of the 3D model. As shown axial tool 242 includes 3 axes axial (A), sagittal (S), coronal (C). Though shown with the axes extending just to a common center point the axes extend through to the related dot 244 opposite the dot 246 with the lettering. By selecting any of the lettered or unlettered dots the 3D model be rotated automatically to the view along that axis from the orientation of the dot 244 or 246. Alternatively, any of the dots 244, 246 may be selected and dragged and the 3D model will alter its orientation to the corresponding viewpoint of the selected dot. In this way the axial tool 242 can be used in both free rotation and snap modes.

A single axis rotation tool 248 allows for selection of just a single axis of the three axes shown in the orientation compass 240 and by dragging that axis in the single axis rotation tool 248, rotation of the 3D model 202 is achieved about just that single axis. Which is different than the free rotation described above, where rotation of one axis impacts the other two depending on the movements of the pointing device.

A 3D model orientation tool 250 depicts an indication of the orientation of the body of a patient relative to the orientation of the 3D model 202. A reset button 252 enables the user to automatically return the orientation of the 3D model 202 to the expected surgical position with the patient lying on their back.

A zoom indicator 254 indicates the focus of the screen. By default, the inner white rectangle will be the same size as the outer grey rectangle. As the user zooms in on the 3D model 202, the relative size of the white rectangle to the grey indicates the level of zoom. In addition, once zoomed in, the user may select the white rectangle and drag it left or right to pan the view of the 3D model displayed in the user interface 200. The inner white rectangle can also be manipulated to adjust the level of the zoom. The plus and minus tags can also be used to increase or decrease the level of zoom.

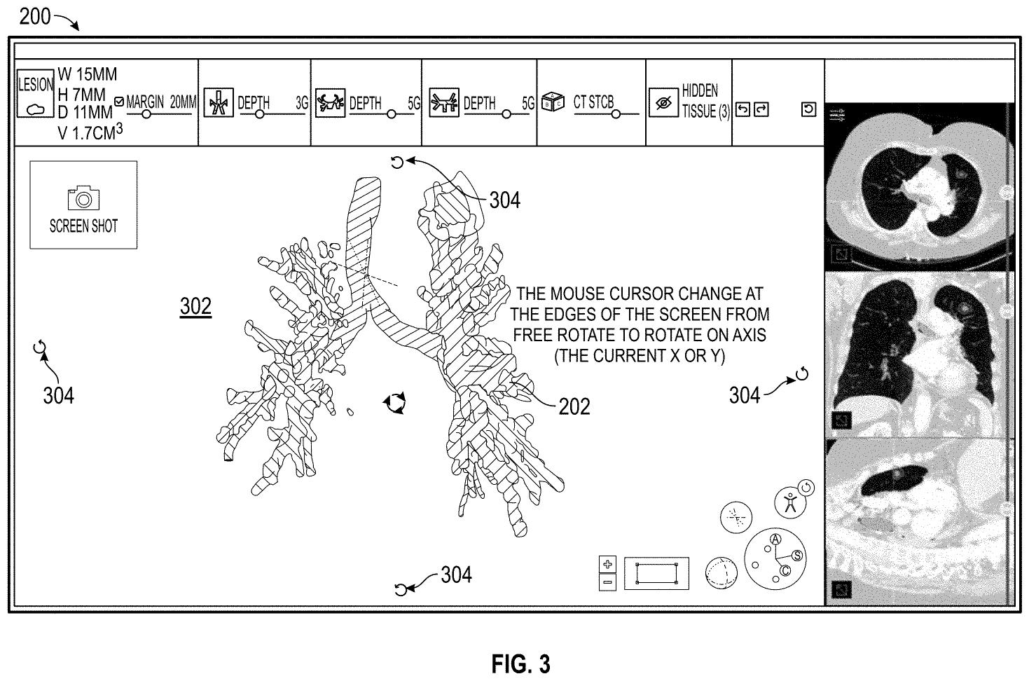

FIG. 3 depicts a further aspect of control of the user interface 200 and particularly the model 202 displayed therein. As shown in FIG. 3, another way to rotate the 3D model 202 in a specific axis is to move the pointing device to the edges of the screen. When this is accomplished an overlay 302 is depicted showing four rotational cues 304. Selecting one of these rotation cues 304 will cause the 3D model 202 to rotate. Additionally, moving the model (i.e., pan) can also be accomplished in this overlay. Further, the pointing device may be used to identify a new spot on or near the 3D model 202 about which to rotate the 3D model 202.

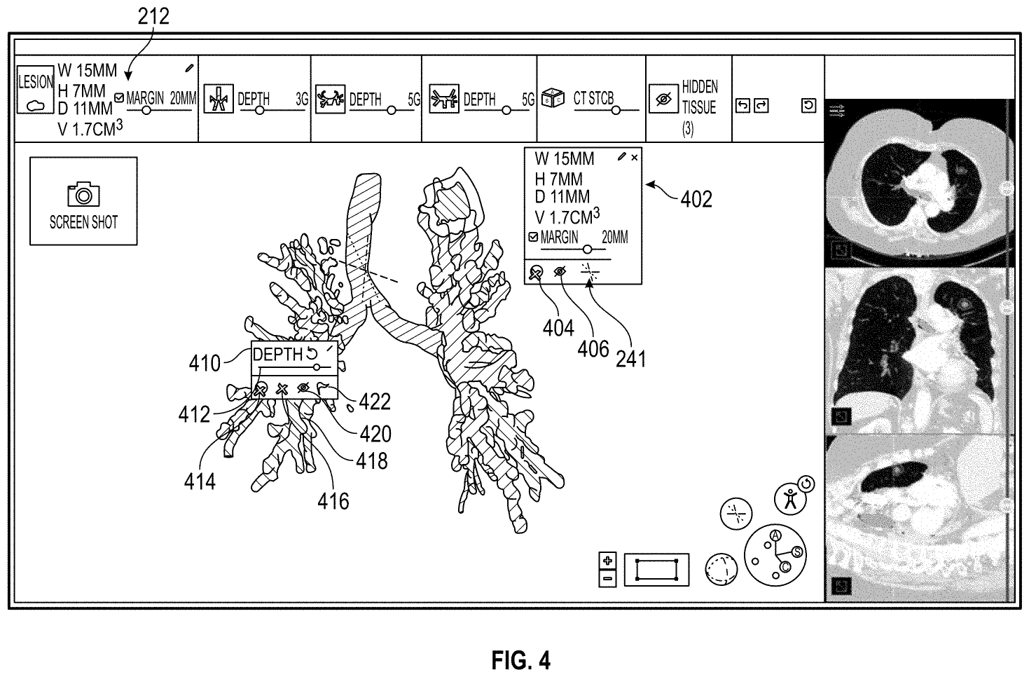

FIG. 4 depicts further features of the thoracic surgery planning tool. When a user selects the tumor 210, the menu 402 is displayed. As an initial matter, the menu 402 displays the same information as the tumor tool 212. Specifically, the menu 402 may display the dimensions and volume of the tumor. The menu 402 also allows for adjusting the size of the margin around the tumor 210 and elimination of the margin all together.

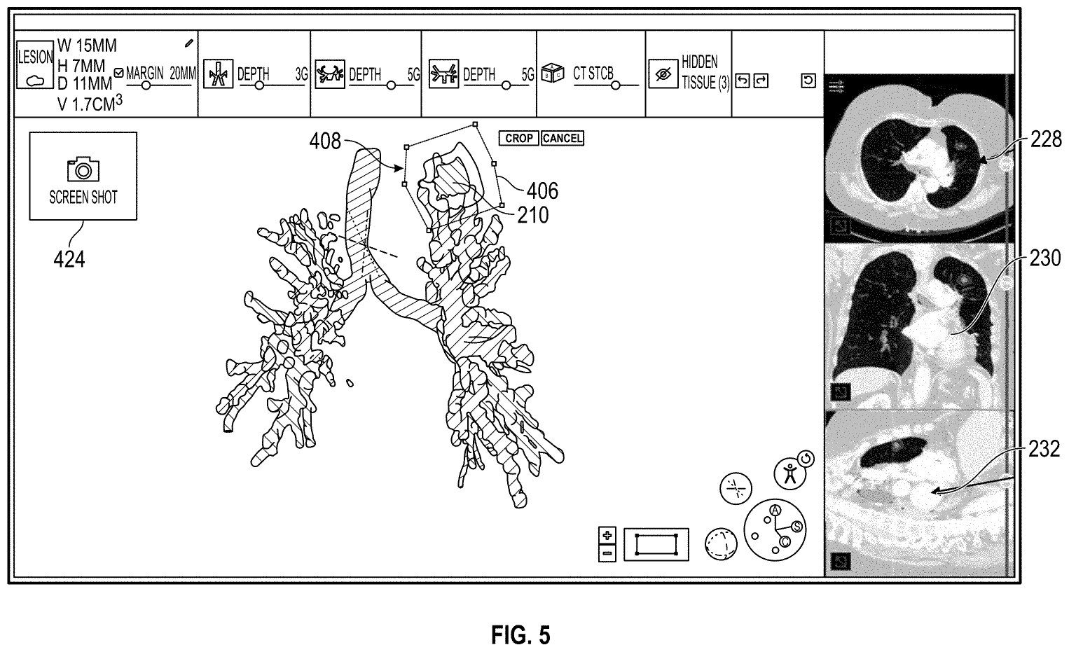

A crop tool 404 is also provided for in the menu 402. When selected the crop tool defines a region 406 around the tumor 210 as shown in FIG. 5. This region 406 is defined by a series of line segments 408. The user is able to select these line segments 408 to adjust the region 406 around the tumor 210. Once satisfied with the placement of the line segments 408 to define the region 406, the user may select the “crop” button. This removes from the 3D model all tissue that is either not found within the region 406 or is not part of an airway or blood vessel, which passes through the region 406. As with the generation selection tools, described above, the effect of this cropping is that not only are the blood vessels and airways that are within the region 406 displayed so the user can observe them and their relation to the tumor 210, but also displayed are the airways and blood vessels which lead to the airways and blood vessels that are within the region 406.

One of the benefits of this tool is to be able to identify the root branches of the airways and blood vessels leading to the tumor 210. This is made possible by removing all of the clutter caused by the other objects (e.g., airways and blood vessels) of the 3D model that are not related to the cropped region. This allows the user to consider the airways and blood vessels leading to the tumor 210 and determine which segments are implicated by the tumor 210 and which airways and blood vessels might need resection in order to achieve a successful segmentectomy. In this manner the clinician can adjust the size of the margin to identify the relevant blood vessels and airways to minimize the area for resection.

The region 406 may be depicted in the CT image slices 228, 230, 232. Similarly, the tissue that has been cropped from the 3D model may also be cropped in the CT image slices. Further, the tissue that is hidden by the crop selection may not be completely hidden but may be ghosted out to limit the visual interference but leave the clinician able to ascertain where that structure is in the 3D model 202.

FIG. 4 depicts two additional features in the menu 402. One feature is a hide tissue button 408 which when selected hides the tumor 210 and any tissue that is within the margin. Further the anchoring tool 241 is also displayed in the menu allowing selection and placement of the anchor point for the orientation compass 240, as described above.

A second menu 410 may be displayed by the user using the pointing tool to select any location within the 3D model 202. The menu 410 includes a depth slider 412 which is enabled by selecting a button 414 shaped like a palm tree allows the user to change the number of generations related to a tissue at the selected point. This allows for local decluttering around the point selected. Additional features in menu 410 include a clip button 416 which provides an indication of the tissue to be excised in the surgical procedure. By selecting the clip button 416, the user may then use the pointing device to select a location on the 3D model 202. A resection line 418 is drawn on the model at that point and the portions of the 3D model to be resected are presented in a different color. A hide tissue button 420 allows for the selection of tissue using the pointing device and hiding the selected tissue from view to again assist in decluttering the 3D model. A flag button 422 allows for placement of a flag at a location in the 3D model with the pointing device and for the insertion of notes related to that flag.

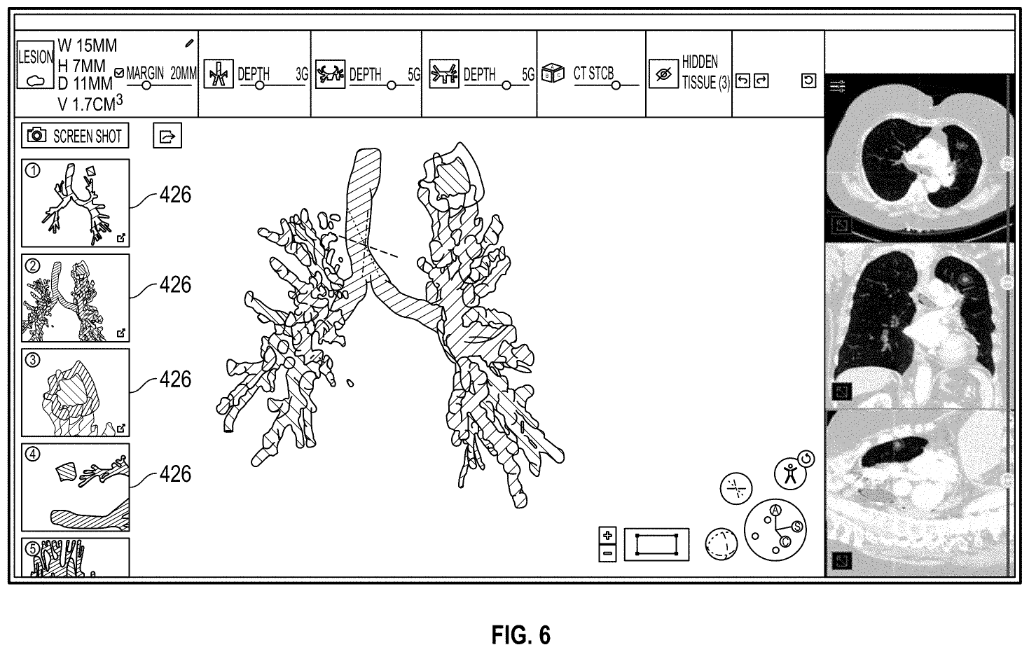

FIGS. 5 and 6 depict a further aspect of the thoracic surgery planning tool in UI 200. Through any of the manipulations described above where tissue is hidden or cropped or the 3D model 202 is rotated a screenshot may be taken by placing the pointing device on the screen shot icon 424. This may be done many times during the thoracic surgery planning as shown in FIG. 6 with screenshots 426 depicted along the left-hand margin of the UI 200. Each of these screen shots shows some prior manipulation of the 3D model 202. For example, screenshot 1 shows just the airways and the tumor 310. In contrast, screenshot 3 shows a zoomed in image of the tumor 210 and related vasculature. Selection of one of these screenshots reverts the 3D model 202 to the model as it appeared in the screenshot 426. In this way during a procedure the clinician can request that a particular screen shot 426 is displayed to refresh their recollection of the expected tissue in a given area or to assist them in identifying the tissue in-vivo so they can proceed with a given resection with confidence that they are cutting or stapling in the correct location. Or that they have accounted for all of the vasculature related to a particular resection prior to making any cuts or stapling. The clinician can arrange these screen shots so that they follow the intended procedure and the information that the clinician seeks to have available during different portions of the procedure. This also allows for a clinician to plan multiple resections and to store each of those plans for multiple tumors in one set of lungs. Further, when one screen shot is selected for viewing, it can be further edited, and this further edited screenshot can be saved separately or used to update the screenshot.

Though described generally herein as a thoracic surgical planning, the software applications described herein are not so limited. As one example, the UI 200 may be shown herein in the surgical room on one or more monitors. The clinician may then direct surgical staff to select screenshots 426 so that the clinician can again observe the 3D model 202 and familiarize themselves with the structures displayed in the screen shot 426 to advise them on conducting further steps of the procedure.

In accordance with another aspect of the disclosure, the UI 202 may be displayed as part of an augmented reality. Further, they may be displayed in an augmented reality (AR) or virtual reality (VR) systems. For example, the UI 200, and particularly the 3D model 202 may be displayed on a headset or goggles worn by the clinician. The display of the 3D model 202 may be registered to the patient. Registration allows for the display of the 3D model 202 to be aligned with the physiology of the patient. Again, this provides greater context for the clinician when performing the procedure and allows for incorporating the plan into the surgical procedure. Alternatively, the UI 200 and the 3D model 202 may be projected such that it appears on the patient such that the 3D model 202 overlays the actual tissue of the patient. This may be achieved in both open and laparoscopic procedures such that the 3D model provides guidance to the clinician during the procedure. As will be appreciated, such projection requires an image projector in the surgical suite or associated with the laparoscopic tools.

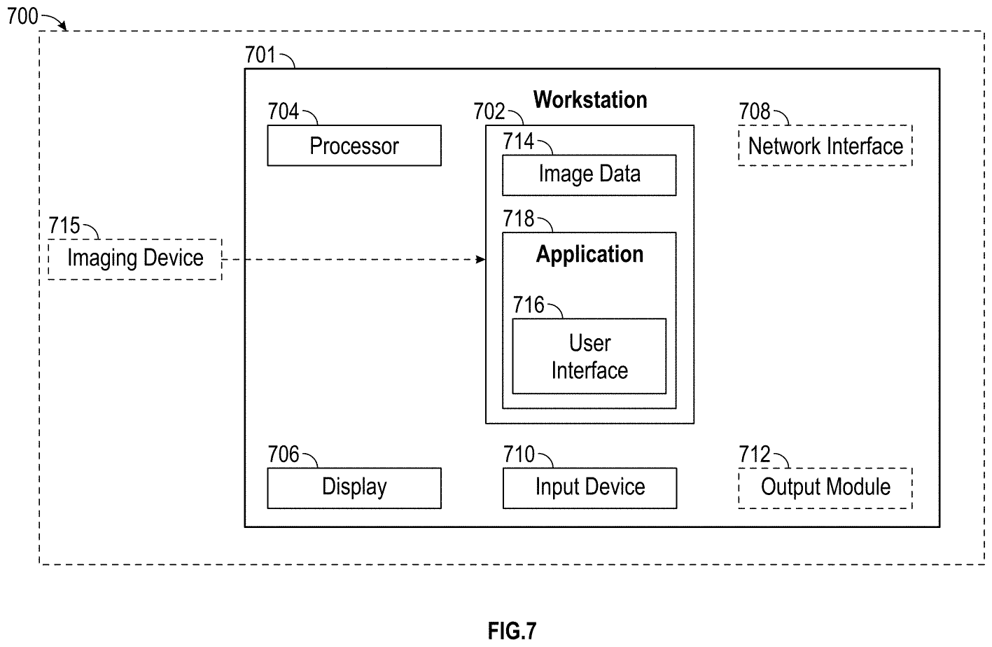

Reference is now made to FIG. 7, which is a schematic diagram of a system 700 configured for use with the methods of the disclosure. System 700 may include a workstation 701, and optionally an imaging device 715 (e.g., a CT or MM imaging device). In some embodiments, workstation 701 may be coupled with imaging device 715, directly or indirectly, e.g., by wireless communication. Workstation 701 may include a memory 702, a processor 704, a display 706 and an input device 710. Processor or hardware processor 704 may include one or more hardware processors. Workstation 701 may optionally include an output module 712 and a network interface 708. Memory 702 may store an application 718 and image data 77. Application 718 may include instructions executable by processor 704 for executing the methods of the disclosure.

Application 718 may further include a user interface 716 such as UI 200 described in detail above. Image data 714 may include the CT image scans or Mill image data. Processor 704 may be coupled with memory 702, display 706, input device 710, output module 712, network interface 708 and imaging device 715. Workstation 701 may be a stationary computing device, such as a personal computer, or a portable computing device such as a tablet computer. Workstation 701 may embed a plurality of computer devices.

Memory 702 may include any non-transitory computer-readable storage media for storing data and/or software including instructions that are executable by processor 704 and which control the operation of workstation 701 and, in some embodiments, may also control the operation of imaging device 715. In an embodiment, memory 702 may include one or more storage devices such as solid-state storage devices, e.g., flash memory chips. Alternatively, or in addition to the one or more solid-state storage devices, memory 702 may include one or more mass storage devices connected to the processor 704 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 704. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 701.

Application 718 may, when executed by processor 704, cause display 706 to present user interface 716. An example of the user interface 716 is UI 200 shown, for example, in FIGS. 2-7.

Network interface 708 may be configured to connect to a network such as a local area network (LAN) consisting of a wired network and/or a wireless network, a wide area network (WAN), a wireless mobile network, a Bluetooth network, and/or the Internet. Network interface 708 may be used to connect between workstation 701 and imaging device 715. Network interface 708 may be also used to receive image data 714. Input device 710 may be any device by which a user may interact with workstation 701, such as, for example, a mouse, keyboard, foot pedal, touch screen, and/or voice interface. Output module 712 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

Though the systems described hereinabove are very useful for planning of a thoracic surgeries, the 3D model information employed in such a thoracic surgery planning system must first be established with confidence. There are a number of methods of generating a 3D model from a CT or Mill data set. Some of these methods employ various neural networks, machine learning, and artificial intelligence (AI) to process the image data set from for example a CT scan and to recognize the patterns to create a 3D model. However, due to the highly overlapping nature of the vasculature, and the limits of image processing, manual methods of analyzing the data set and generating the 3D model or updating/correcting the 3D model are desirable. A further aspect of the disclosure is directed to a tool 800 that allows for expert annotation of pre-procedure images (e.g., a CT scan or an MRI data set) to define all or a portion of the vasculature of the patient, particularly the vasculature around the lungs and heart in the thoracic cavity.

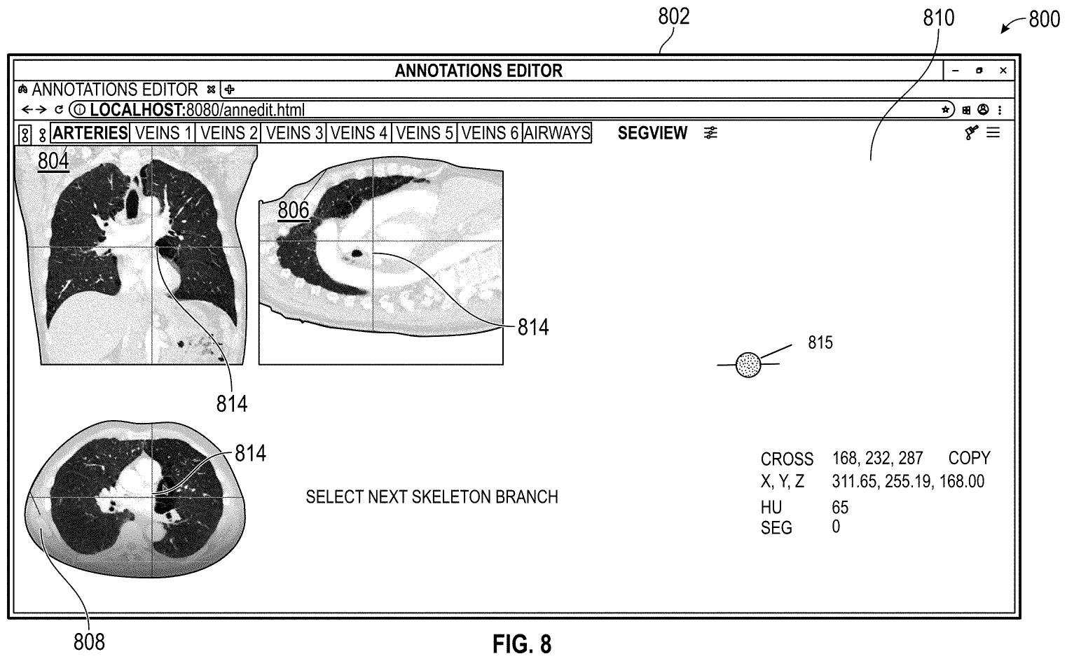

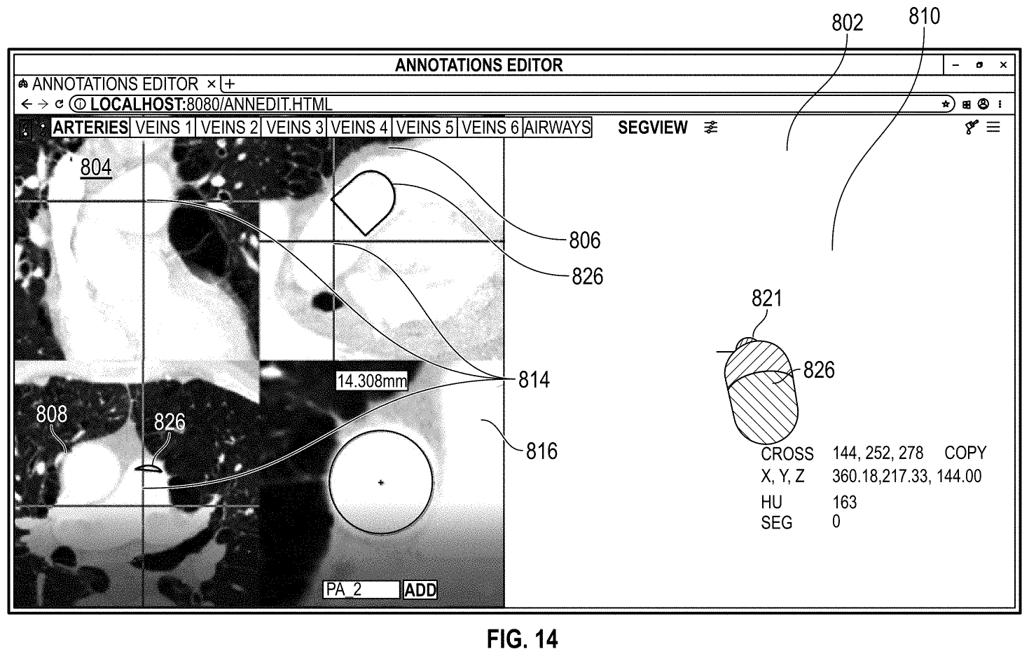

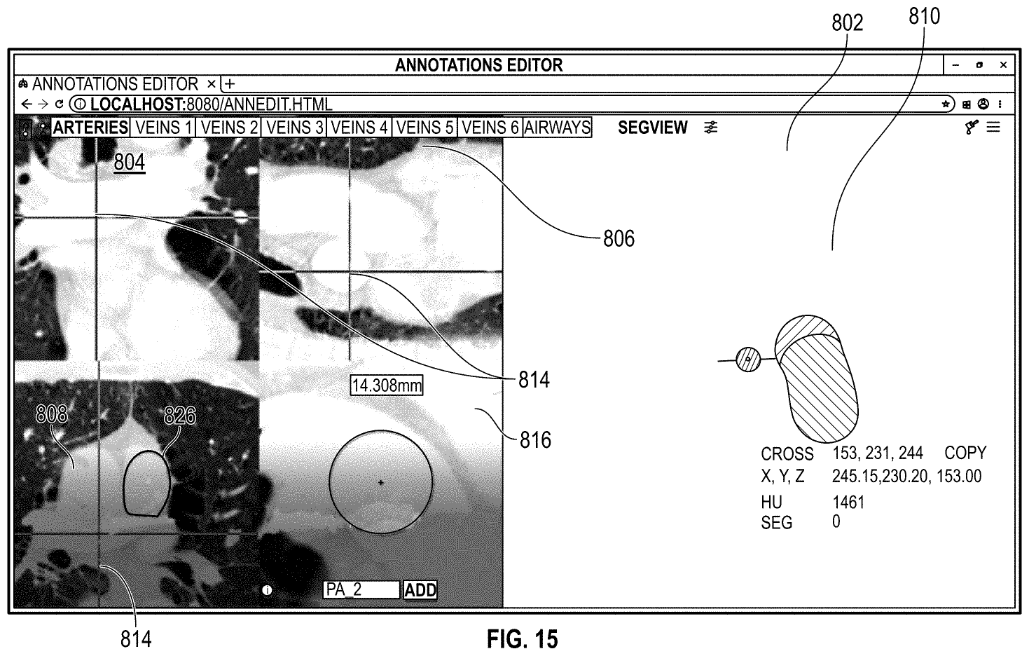

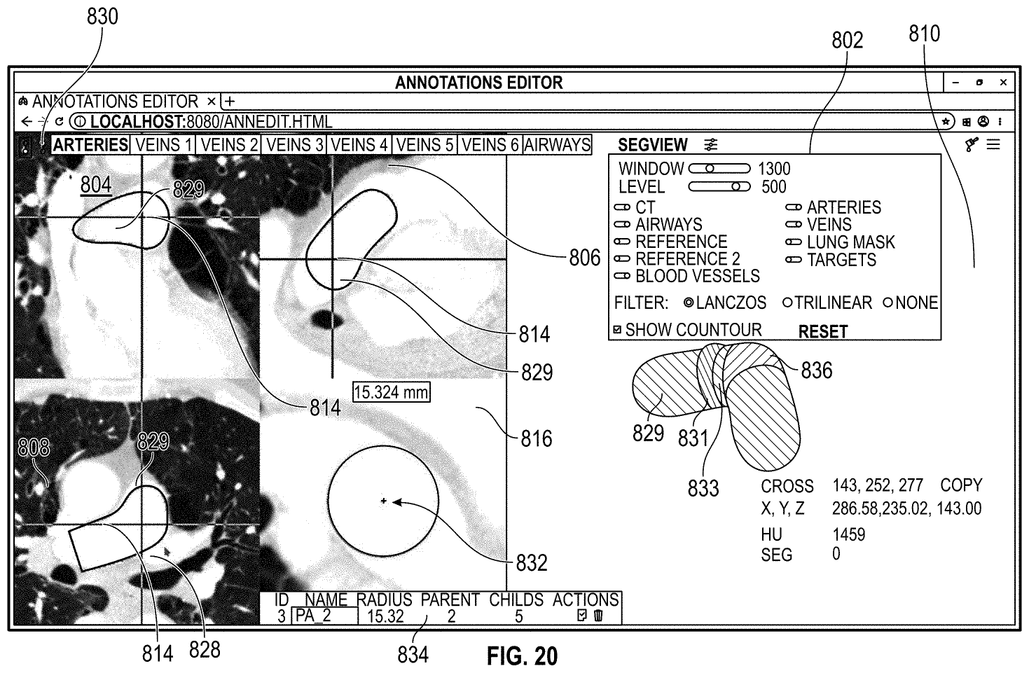

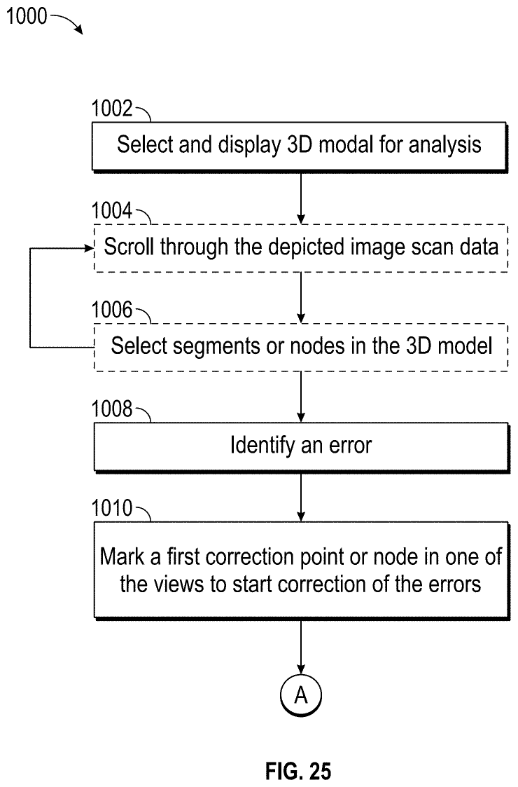

FIGS. 8 and 10-26 are screen shots taken from the tool 800. The tool 800, a software application 718 running on workstation 701 is configured to display a user interface 802 and allows for the import of the pre-procedure image data set, e.g., a CT scan of the patient. The user interface 802 displays three standard views of a portion of the patient's anatomy in a coronal image window 804, a sagittal image window 806, and an axial image window 808. A three-dimensional view (3D View) window 810, as will be described below is used to depict 3D segments of the selected vasculature in the volume defined by the CT scan data and ultimately the 3D model 850 formed of these interconnected segments as shown in FIG. 26.

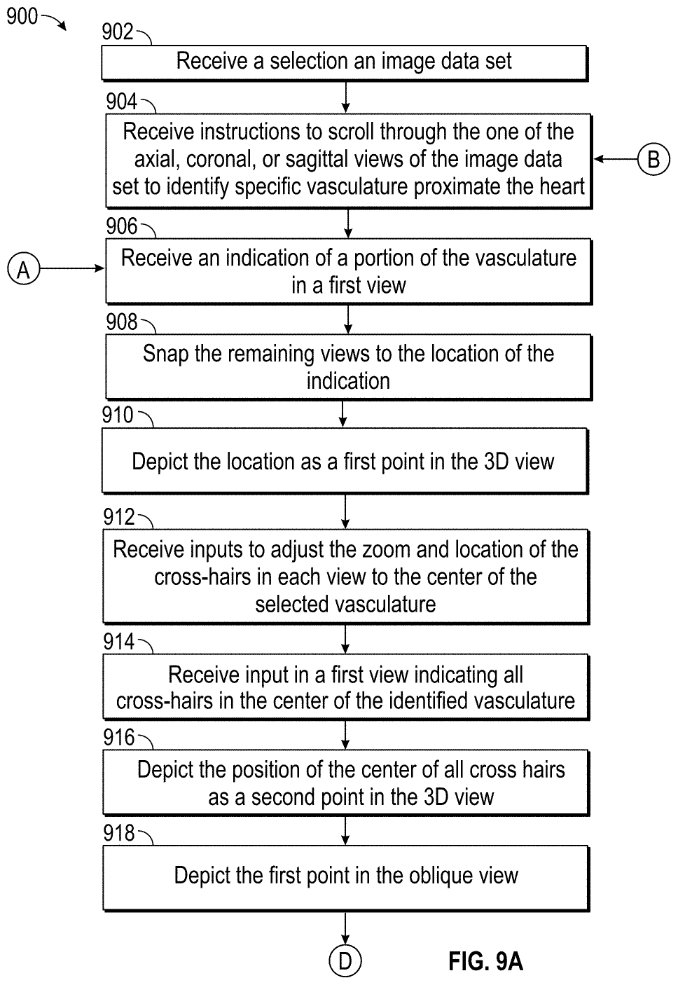

In accordance with one method of use 900 as outlined in FIG. 9, a selects an image data set, e.g., a CT data set for presentation in the UI 802 at step 902. Once selected the tool 800 presents a UI 802 depicted in FIG. 8 on the display 706. The selected image data set has been processed, for example via segmentation techniques, and is presented in three orthogonal views coronal, axial, sagittal, and the UI 802 enables a user to select one of the views and scroll through the views using an input device such as a mouse, touchscreen, keyboard, pen, and others to communicate with the workstation 701 and thus the application tool 800 at step 904. When a user observes a portion of the vasculature in the depicted views 804, 806, 808, for example in the axial view window 808, the user may use an input device such as a mouse, touchscreen, or keyboard to identify the vasculature at step 906, which places crosshairs 814 in the vasculature. The other two views (e.g., the coronal view window 804 and sagittal view window 806) snap to the same location in the image data set at step 908. In general, the three image view windows are linked such that when a cursor hovers over any one of them, scrolling in that view window will result in scrolling in all three view windows. In addition, the identification of any point in the depicted views 804, 806, 808 results in the generation of a first point 815 in the 3D model view 810 at step 910. At any point when the crosshairs 814 are moved, the location of the first point 815 in the 3D space of the 3D model view 810 is moved.

At step 912 the level of zoom and the position of the crosshairs 814 is adjusted to in each view window 804, 806, 808 so that the crosshairs 814 are positioned in the center of the selected vasculature. Once so positioned at step 914 an input is received indicating that all three crosshairs 814 are in the center of the center of the vasculature in the three view windows 804, 806, 808. This input may be for example, clicking again in the view window where the vasculature was originally identified (e.g., axial view window 808, step 904). Following receipt of the input, at step 916 a second point 818 is placed in the 3D view 810 depicting the location of the three crosshairs 814 in the 3D volume of the image scan data. At step 918, the first point 815 is depicted as a cross 817 in an oblique view 816. The oblique view 816 is the view from within the CT image data set from the first point 815 along an axis that would connect with the second point 818.

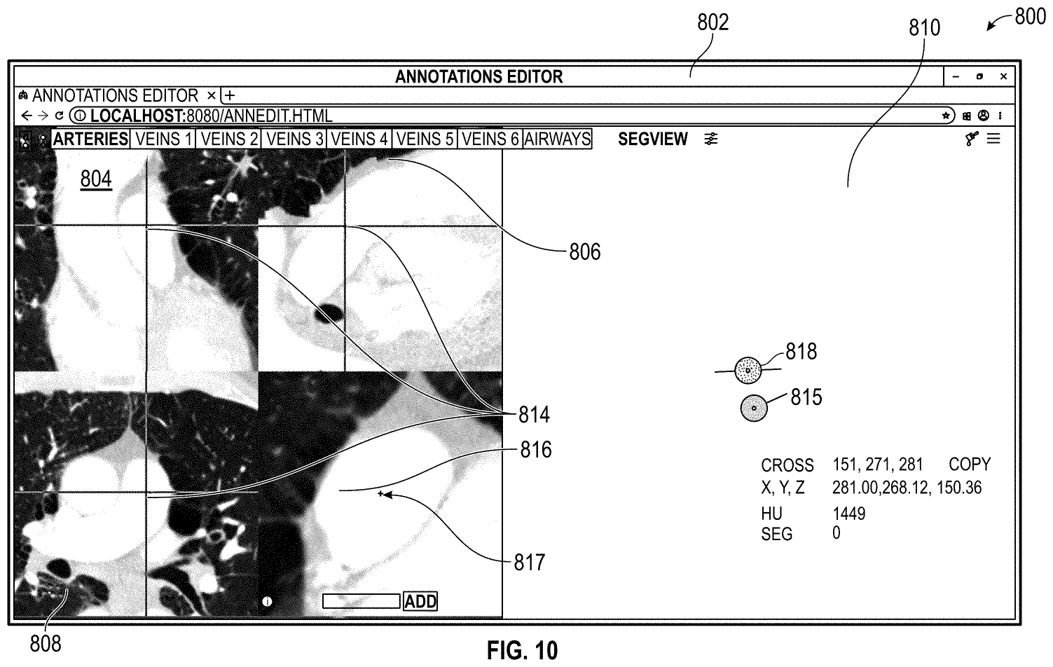

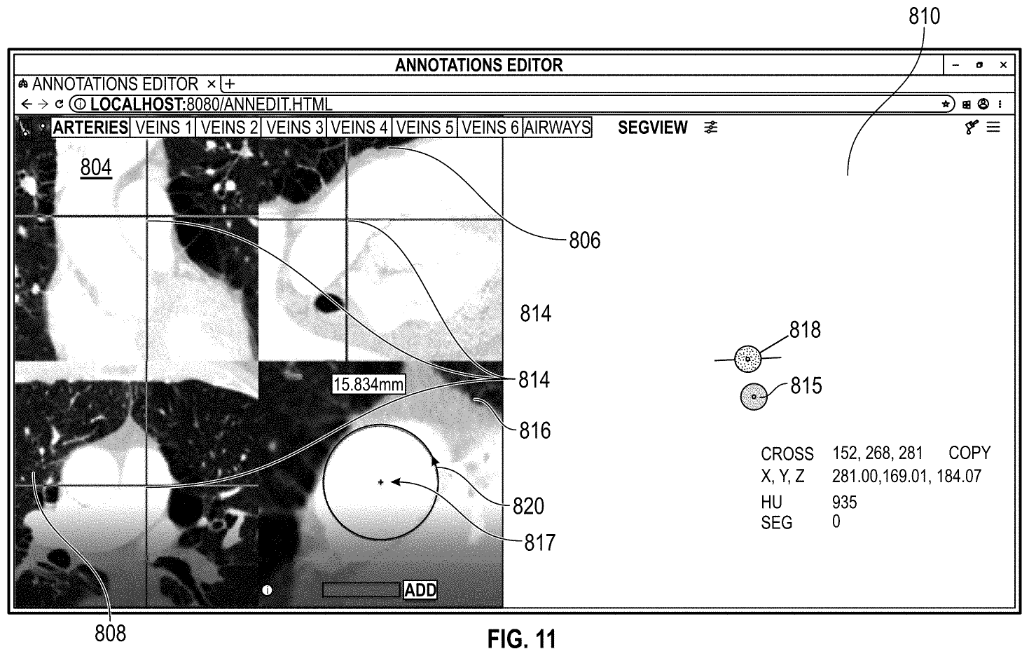

With the first point 815 depicted in the oblique view 816 as a cross 817 as shown in FIG. 11, when the cursor driven by the input device (e.g., a mouse) is placed in the oblique view 816 at step 920, a circle 820 is depicted in the oblique view 816 centered on the crpss 817 in the oblique view at step 922. Inputs are received at step 924 (e.g., via a mouse) to size the depicted circle to match that of the selected vasculature depicted in oblique view 816 (FIGS. 11-12). In addition, the image that is displayed may be moved along its plane to ensure that the circle 920 is centered in the depicted vasculature in that image of the oblique view. As will be appreciated, while in the depicted views 804, 806, 808, moving of the crosshairs is limited to the whole the voxels (integer values), the oblique view 816 is not so limited thus, the oblique view 816 provides much greater granularity of movement to ensure that the circle 820 is centered in the vasculature.

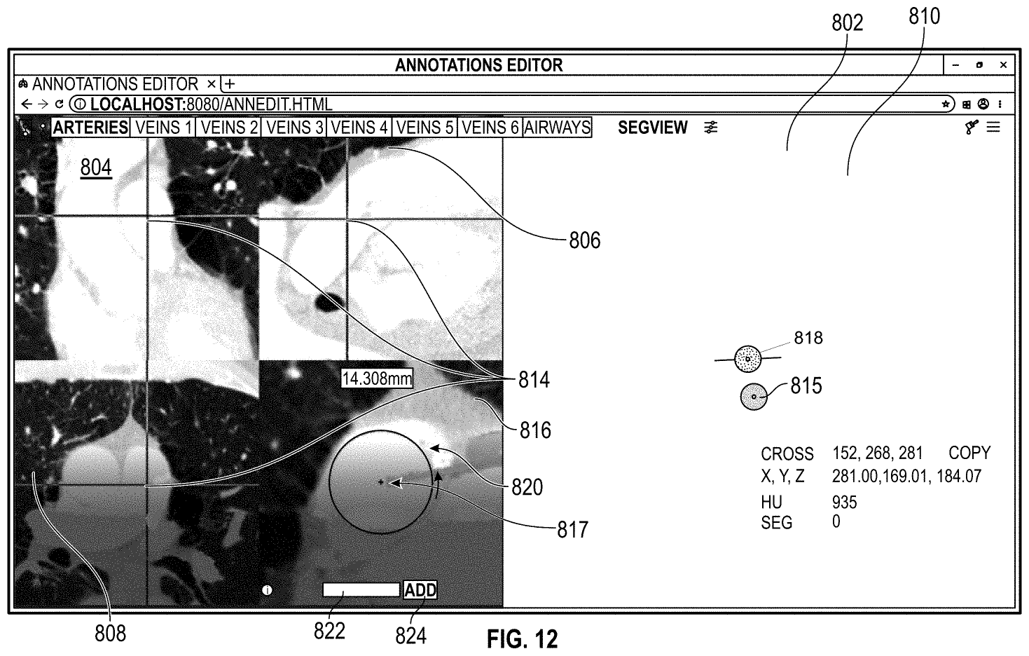

Following sizing of the circle 820, a segment name is optionally added via the input device (e.g., a keyboard) in naming box 822 at step 926 and the add button 824 is selected at step 928 The segment 826 is displayed in the 3D view 810 at step 930 and in the axial, coronal, and sagittal views at step 932 (FIG. 13). The segment 826 is the portion of the selected vasculature from the first point 815 to the second point 818. The segment 826 has the diameter that was defined by the sizing of the circle 820 around the cross 817 depicted in the oblique view 816 at step 924. Thus, the segment 826 has the diameter of the vasculature at the first point 815. The segment 826 is depicted in the 3D view 810 (FIG. 13), with a node 821 being depicted with a contrasting color or texture to the rest of the segment 826 at the location of the second point 818. The contrasting color or texture throughout the 3D model generation process to depict a node 821 that is the end point of a segment 826 just generated and is an indicator of the direction of modeling within the vasculature. The segment 826 is also depicted in each of the view window 804, 806, 808 on the images depicted there, and the displayed position in the view windows 804, 806, 808 and the 3D view is updated to be centered on the second point 818.

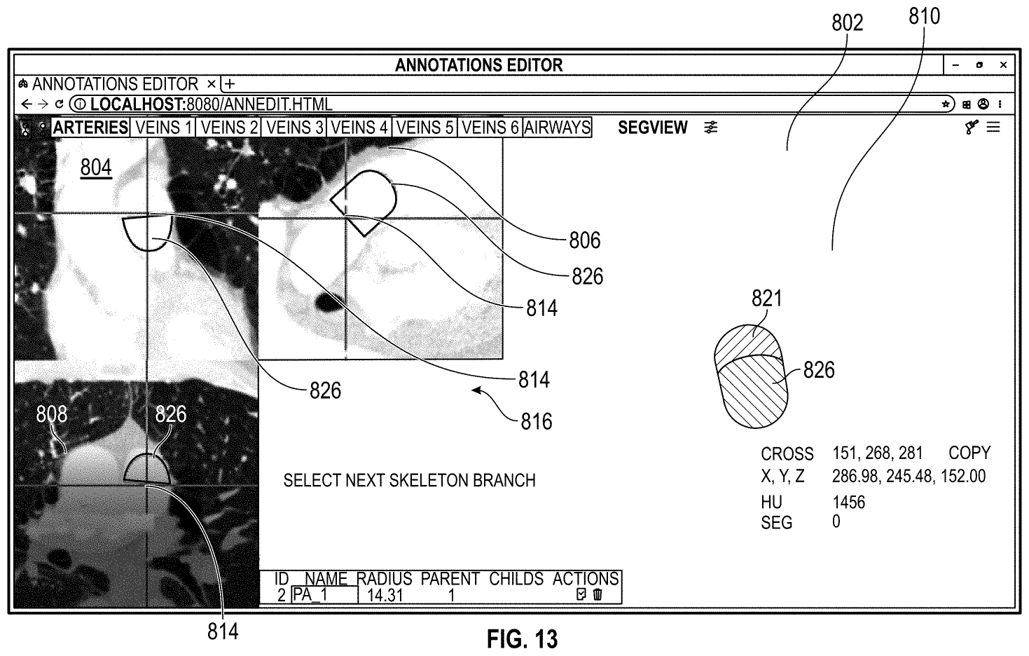

Following depiction of the segment 826 in the 3D view 810 the application asks a user to determine whether all segments have been marked and if not to user is directed in step 936 to scroll the images similar to step 904, but within branch of the selected vasculature as the first segment 826. The process 904-936 repeats to identify a next point and to generate the next segment to depict in the 3D view 810 and the view windows 804, 806, 808 (Step 938) as depicted in FIGS. 14-17.

Those of skill in the art considering the process will understand that in a second segment between a second and a third point, the diameter of the segment will be based on the diameter of the second point 818. If the diameter of the vasculature at the second point is similar to the first point, the segment will be substantially cylindrical. If the diameter at the second point is less than the diameter of the first point, then the segment 826 may be adjusted to reflect the change in diameter that decreases from the first point to the second. This process continues with the subsequent segment updating the diameter of the preceding segment until all segments of the selected vasculature have been marked and depicted in the 3D view 810.

If it is believed that all segments of the originally identified vasculature (Yes at step 934) the method moves to step 938 where the user must determine whether all the vasculatures have been identified and incorporated into the 3D model. For example, if only the arteries extending from the right pulmonary artery has been identified and modeled, the answer to question 940 is no, and the process returns to step 904 so that additional vasculature may be identified and mapped in the image data set. For example, the user may employ the processes described above to generate a 3D map of the left pulmonary artery. Subsequently, the left inferior pulmonary vein left superior pulmonary vein, the right superior pulmonary vein, and the right inferior pulmonary vein may all be mapped using the processes described above. If the user, viewing the 3D model and considering the image data set believes that all such vasculature has been modeled then the answer to question 930 is yes. The application 718 may receive input from an input device to save the 3D model at step 940 and the process ends. The saved 3D model may be imported as the 3D model 202 and analyzed using the user interface 200 of a thoracic surgery planning system of FIG. 2.

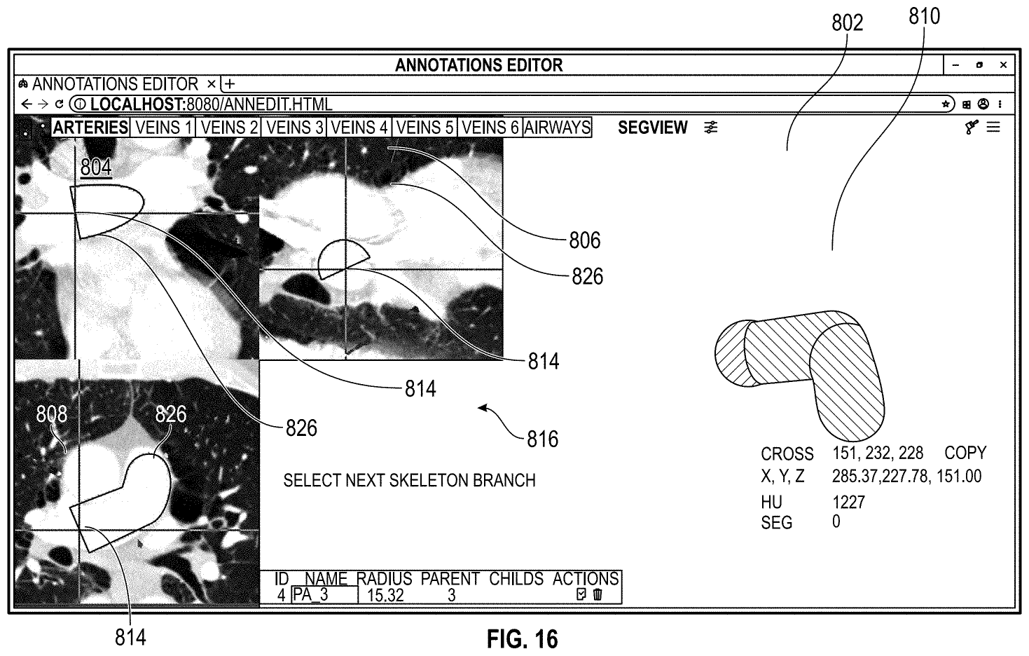

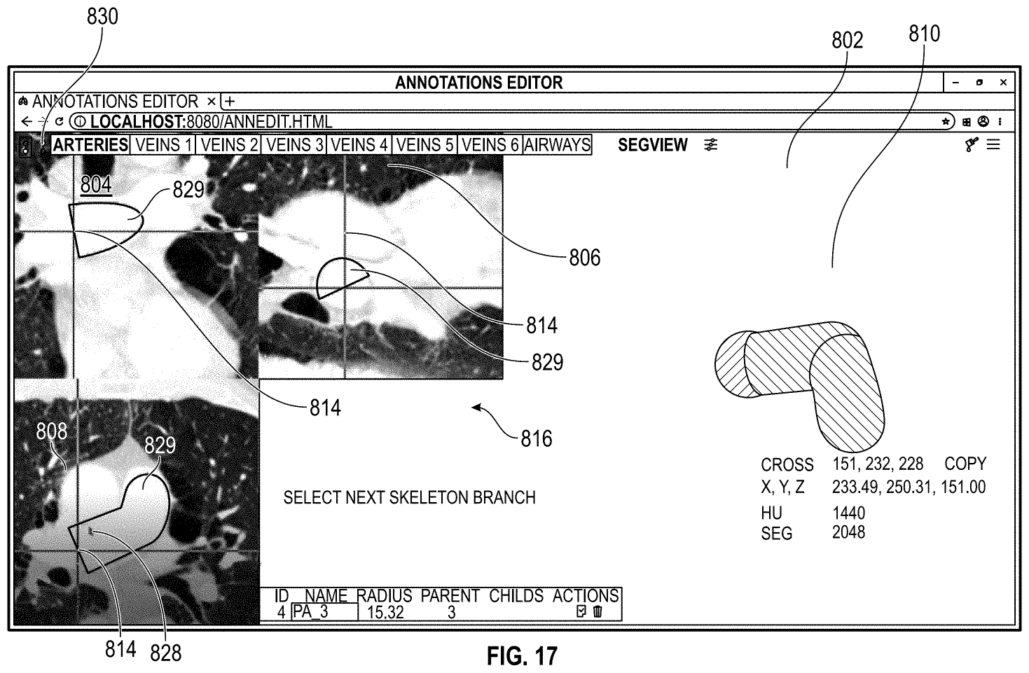

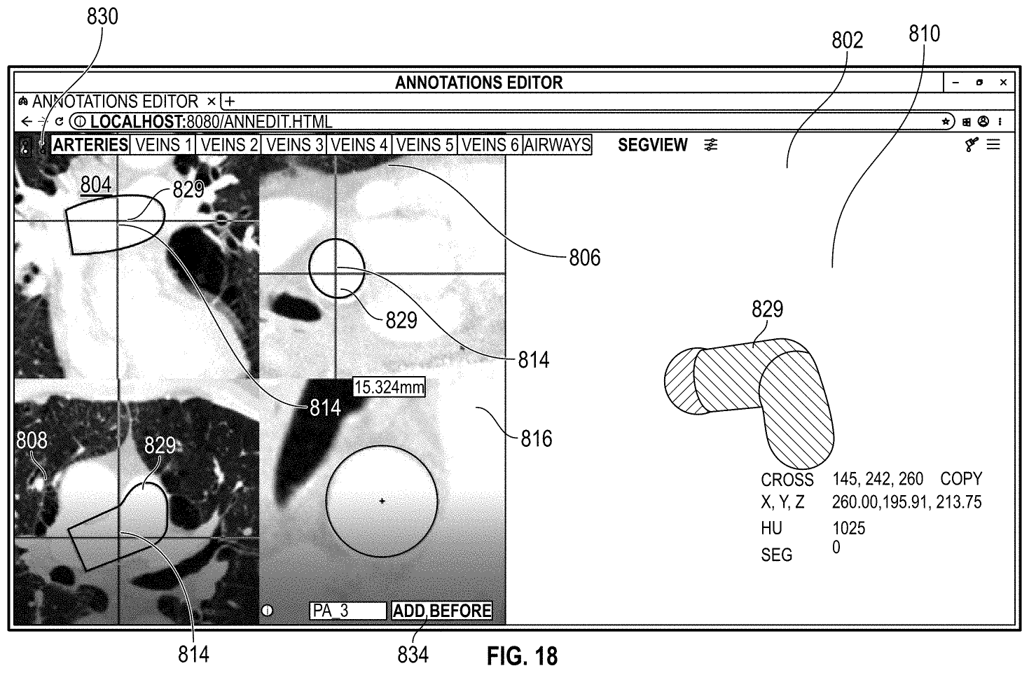

While the forgoing describes the basic functionality, additional functionality of the application depicted in the user interface 802 is available. For example, at any point during the process a previously defined segment may be observed to extend outside the boundaries of the vasculature as depicted by the cursor 828 in FIG. 17 in the axial view window 808. If such an occurrence is observed, the crosshairs 814 are moved to within the segment 829 that requires editing as depicted in FIG. 18. Once so positioned, as shown in the view windows 804, 806, 808, an “insert segment before” button 830 may be selected using the input device. This returns the process to step 906, and the steps 906-930 are undertaken to define a node 831 (FIG. 19) at a point 832 within the previously defined segment 829. The identification of point 832 and scrolling through the at least one of view windows 804, 806, 808 to selection of a second point (not shown) and the sizing of the diameter in the oblique view 816 in FIG. 18 is used to generate a new segment 833 (FIG. 19). After this length and diameter is defined, upon arriving at step 932 the name is optionally entered and/or the “add before” button 835 as shown in FIG. 18.

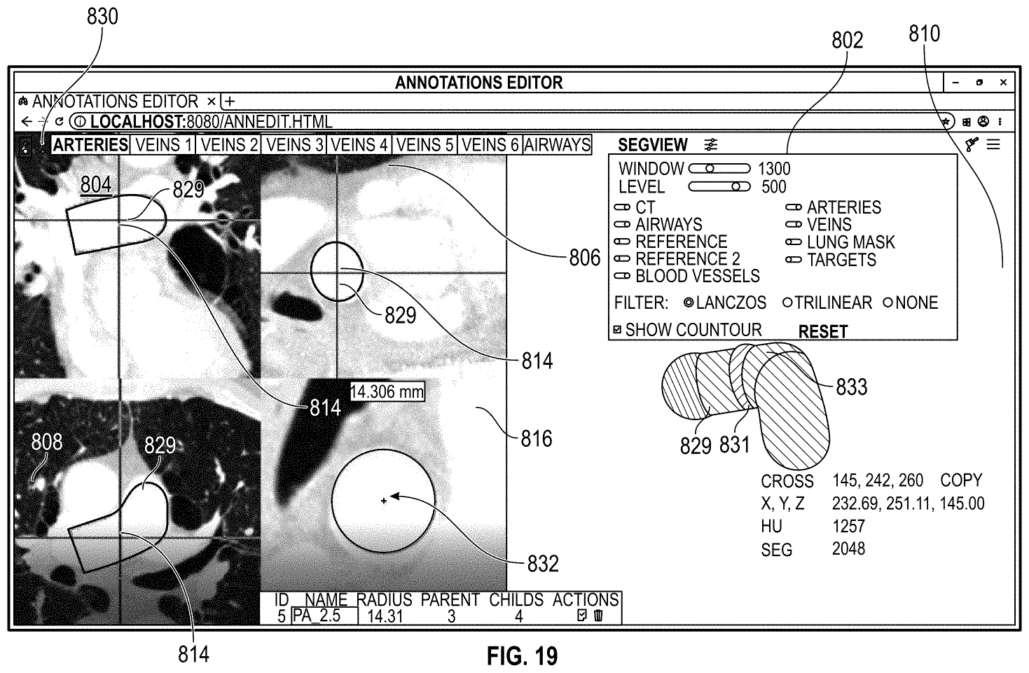

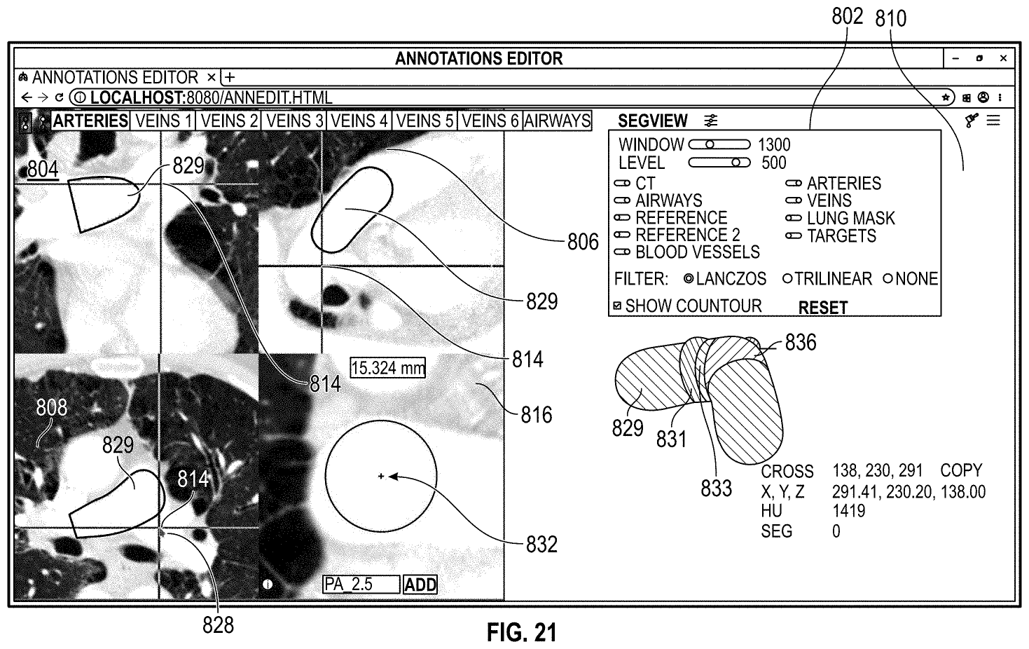

At any point during the modeling, a bifurcation in the vasculature may be identified in the view windows 804, 806, 808. For example, the cursor 828 in FIG. 20 is shown in a branch of the vasculature separate from the segments 829, 833. By scrolling in one of the view windows (e.g., axial view window 808), the user can confirm that this is indeed a branch of the same vasculature, this step is most likely to occur during one of the steps 918 in the method 900, described above. By moving at least one of the vertical or horizontal portions of the crosshairs 814 to a location that is the start of the branching structure (FIG. 20), the tool 800 will identify the closest node 836 in a segment 833 in the direction vertical or horizontal portion of the cross hair 814 was moved. The node 831 at the opposite end of the segment 833 is also identified but in a different color or texture to identify a direction of movement in the modeling. Following the steps 918-934, outlined above, a new segment 838 of the branch vasculature can be defined with a specific length and having a node 840, as depicted in FIG. 22.

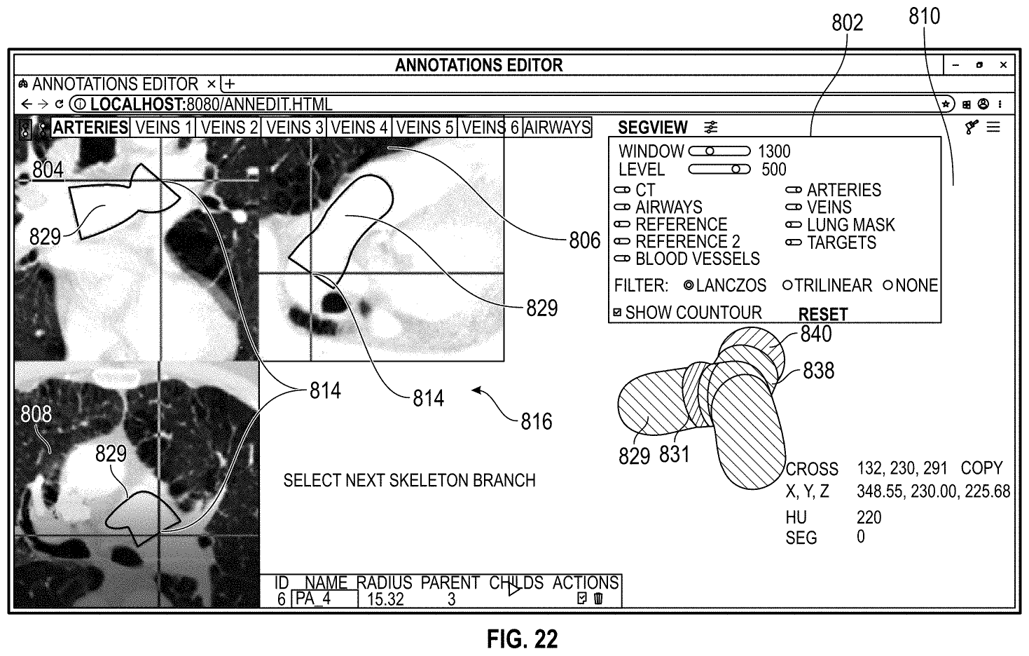

Any node or segment may be selected and rotated in the 3D view 810 as shown by comparison of FIGS. 23-25. Such a selection of a node or segment and rotation will cause a related change to the image view windows 804-808. By utilizing these tools, a user is able to move through the image data set and define the structures of the vasculature in exacting detail. FIG. 23 shows a dropdown menu 850 with some additional functionality that may enable the user to better identify vasculature within the image data set. For example, sliders 852 are provided for adjusting the contrast of the images depicted in the view windows 804, 806, 808, one for adjusting the window and one for adjusting the level. Those of skill in the art will recognize that window refers to the range of greyscale that is displayed and center of the greyscale is the window level. In addition, various references, toggles for arteries and veins, lung mask and targets may be toggled on or off as desired by the user.

Described herein are a variety of interactions with the windows 804-808 and 816. As described herein these are undertaken with a mouse, touchpad, or other pointing device useable with a computing device. Additionally or alternatively, the UI 802 may also receive input via a keyboard or a touchscreen, both of which can be particularly useful when annotating the 3D model or the images. The use of these tools for interacting with the UI 802 ease navigation through the views and the 3D model and enable translation, rotation and zoom of the 3D model.

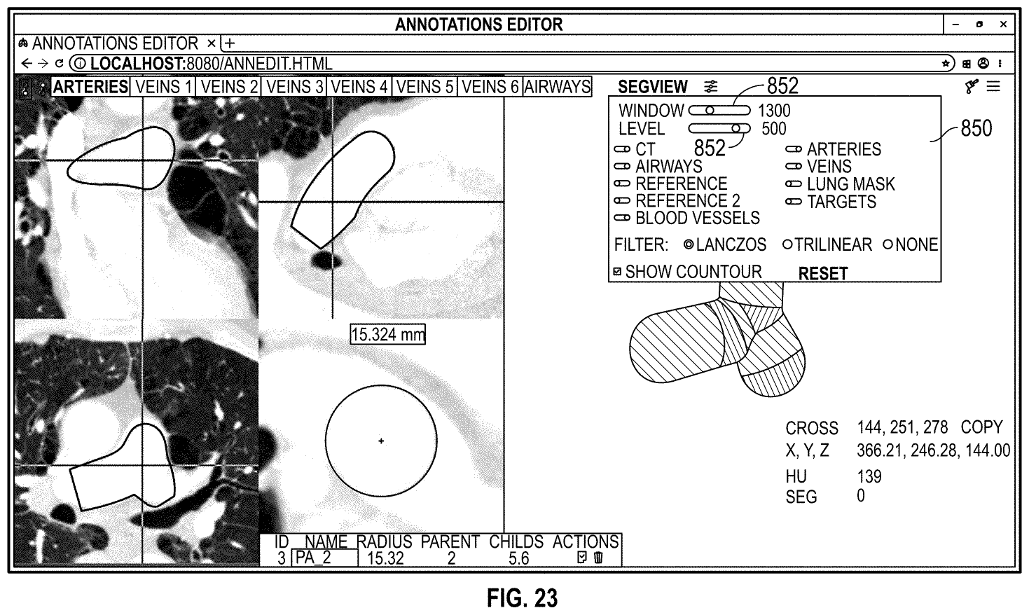

Ultimately the entirety of the image data set and all, or substantially all, of the vasculature in the image data set can be modeled using the method of FIG. 9 to achieve a complete model of the vasculature as depicted in FIG. 24 for importation into a thoracic procedure planning system, such as that depicted in FIG. 2, above, or for other uses.

Though described above with respect to a fully manual operation the instant disclosure is not so limited. In accordance with one aspect of the disclosure, instead of manual modeling of the vasculature, the vasculature in the image scan data may first be automatically processed by an algorithm, neural network, machine learning, or artificial intelligence to produce an initial 3D model. Known techniques for this initial 3D model creation can be employed. These techniques generally use contrast and edge detection methods of image processing to identify the different portions of the vasculature, identify other structures in the images scan data, and make determinations regarding which are veins, arteries, and airways based on a variety of different matters. These systems may also employ connected component algorithms to seek to form boundaries on the identified structures to limit the bleeding of the modeled segments into neighboring but separate vasculature.

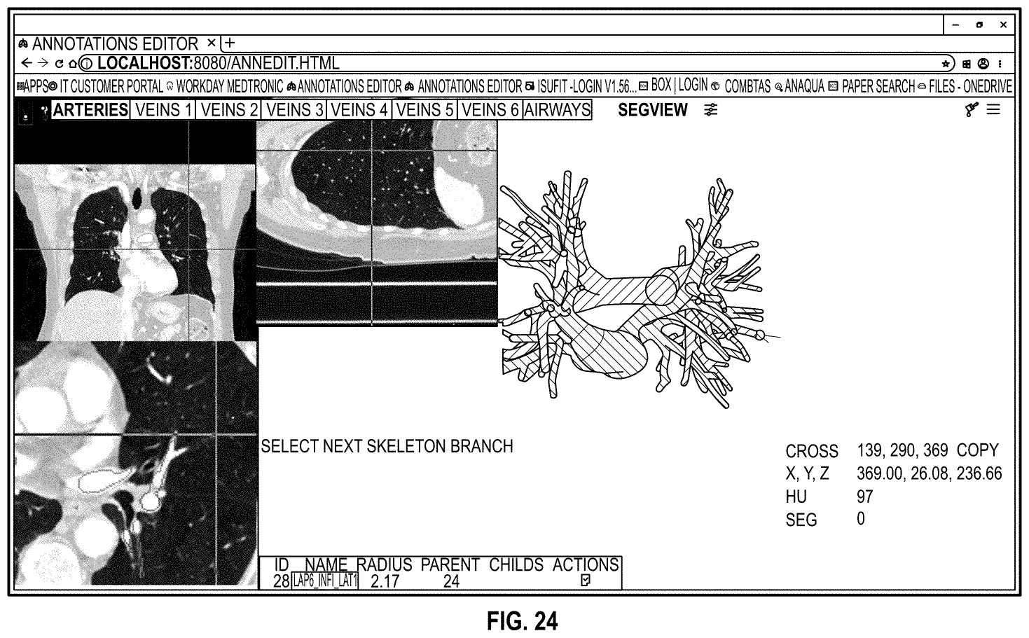

Regardless of the techniques employed, a method of reviewing the 3D model is described with reference to FIG. 25 and method 1000. At step 1002 the 3D model may be selected and displayed in the UI 802. Next the application can receive inputs via an input device such as a mouse, touchscreen, keyboards, etc. to scroll through or control the zoom of the image scan data in the image view windows 804, 806, 808 at step 1004. Additionally or alternatively, the application may receive an input to select segments or nodes in the 3D model at step 1006. Using steps 1004 and 1006 the entirety of the image scan data and the 3D model may be reviewed. At any point, an error may be identified at step 1008. As will be appreciated, using the functionality described above, the application may receive inputs to scroll, zoom or otherwise manipulate the display of the image scan data in the view windows 804, 806, 808 to analyze the error. At step 1010 a first correction point or node is marked in the one of the view windows 804, 806, 808 of the image scan data. Once so marked, the process reverts to step 918 for further manipulation of the image scan data and generation of a corrected segment. This process continues until all of the segments of the 3D model and all of the image scan data has been analyzed and the 3D model corrected where necessary. The corrected 3D model is saved at step 942, as described above. In this manner time can be saved in the creation of the 3D model and confidence in 3D model enhanced by the manual review and correction prior to the 3D models availability for use in the thoracic surgery planning system, described above.

Still further, those of ordinary skill in the art will recognize that the corrected 3D models, and their differences from the 3D models that were automatically generated may be used to train the neural networks, algorithms, AI, etc. to improve the output of the automatic 3D model generation systems.

Another aspect of the disclosure is directed to a partial automation of the process described above. The FIG. 26 is a flow diagram for a semi-automatic method to generate a 3D model or to extend an existing 3D model. The method 1100 requires selecting a starting point in the existing 3D model or the image scan data in the view one of the windows 804, 806, 808 and then scrolling the scan data in to track the blood vessel in the image scan data as displayed all the way to the periphery. Once an end point or a next point in a blood vessel is observed it is selected in the image scan data and using a shortest path algorithm the blood vessel between the selected end point and the starting point is automatically generated and included in the 3D model. As will be appreciated, this method of blood vessel generation for the 3D model greatly increases the speed at which individual blood vessels can be generated and ultimately the 3D model.

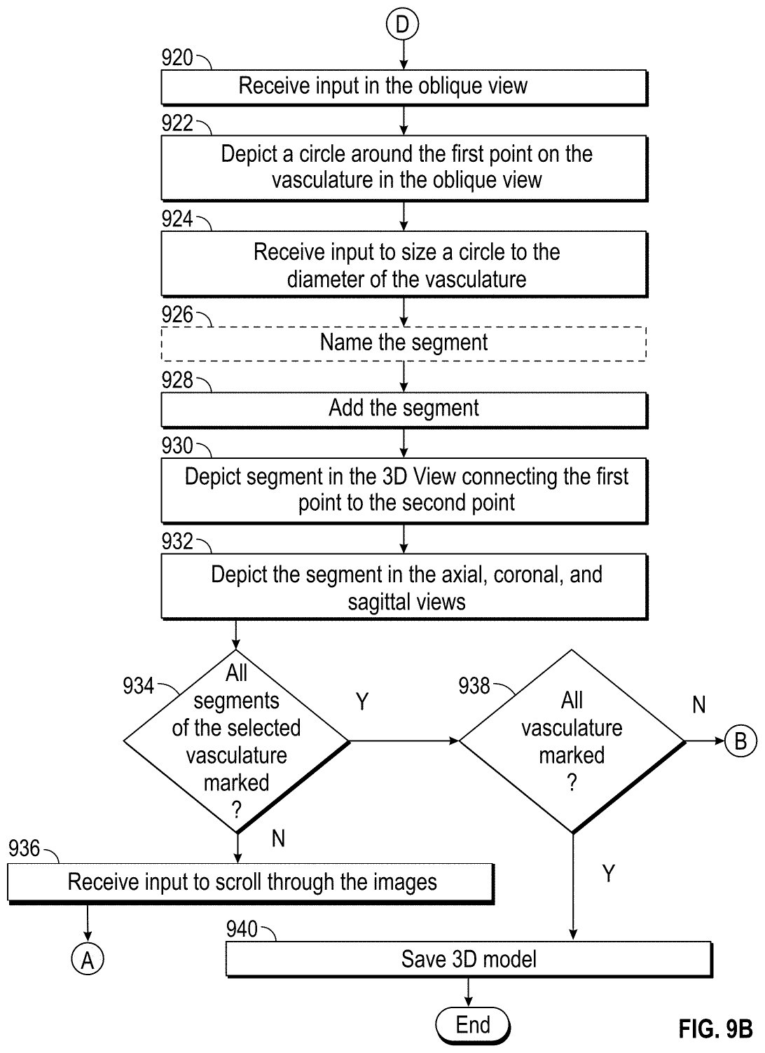

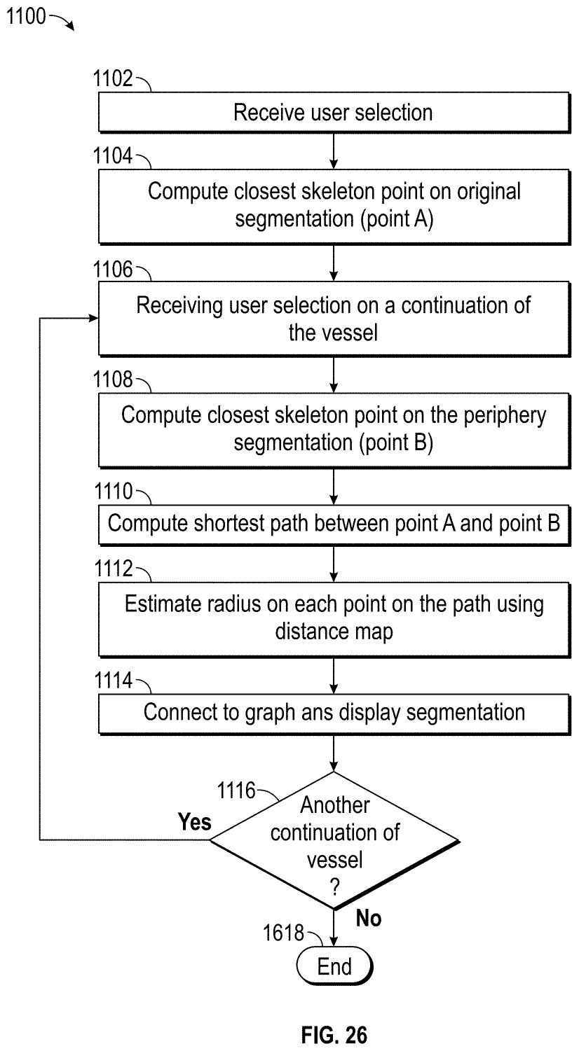

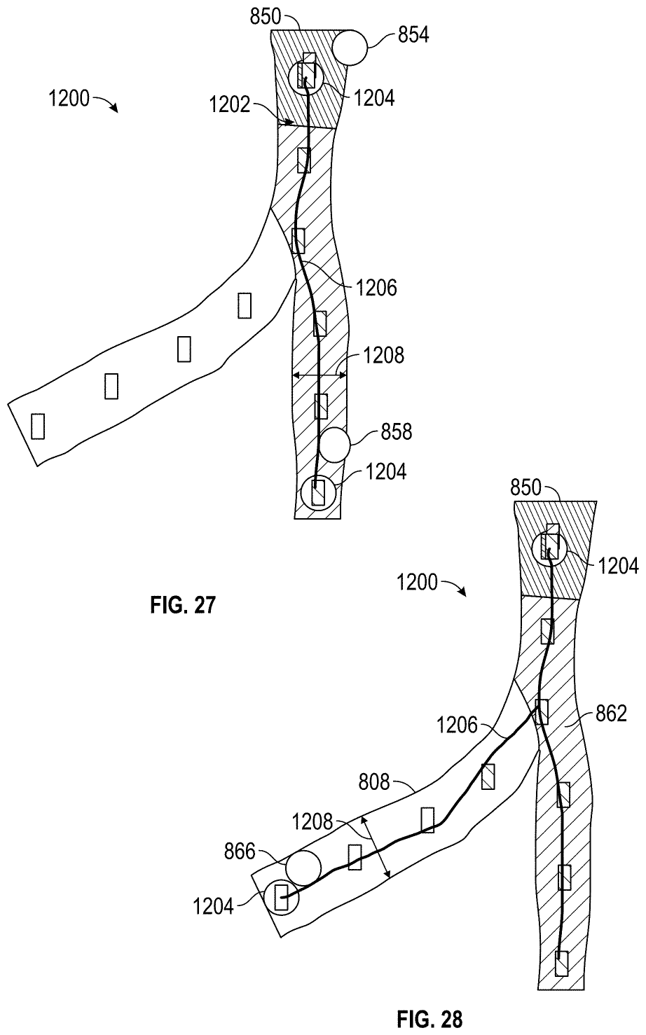

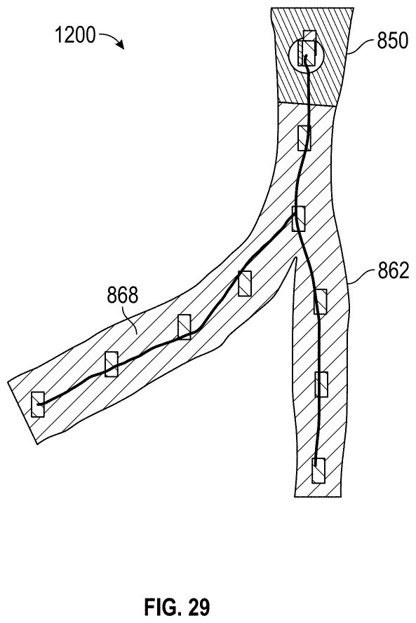

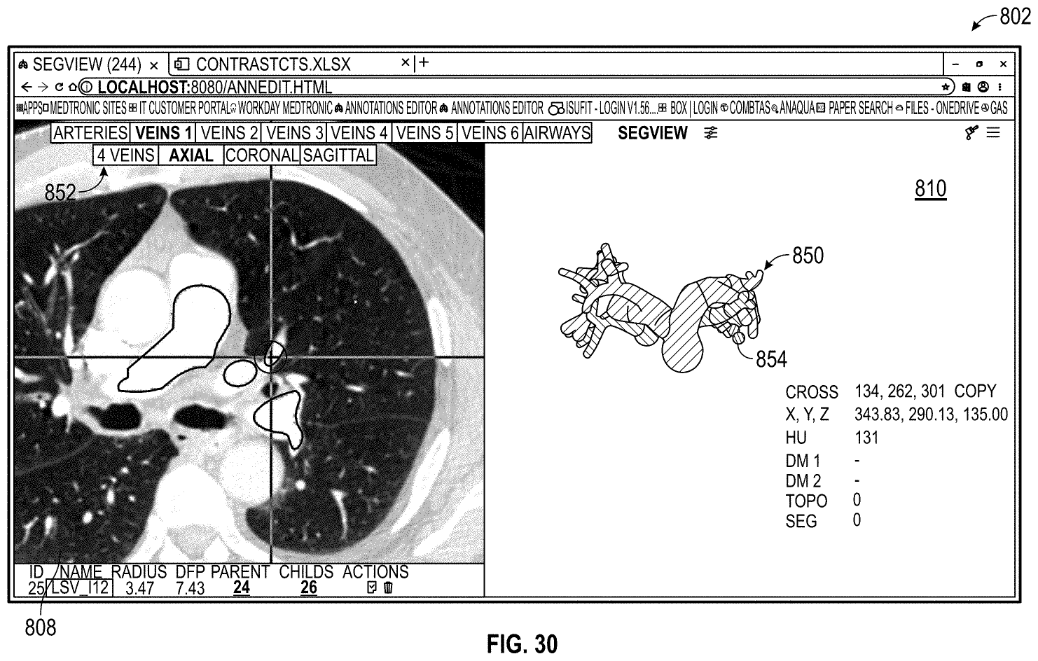

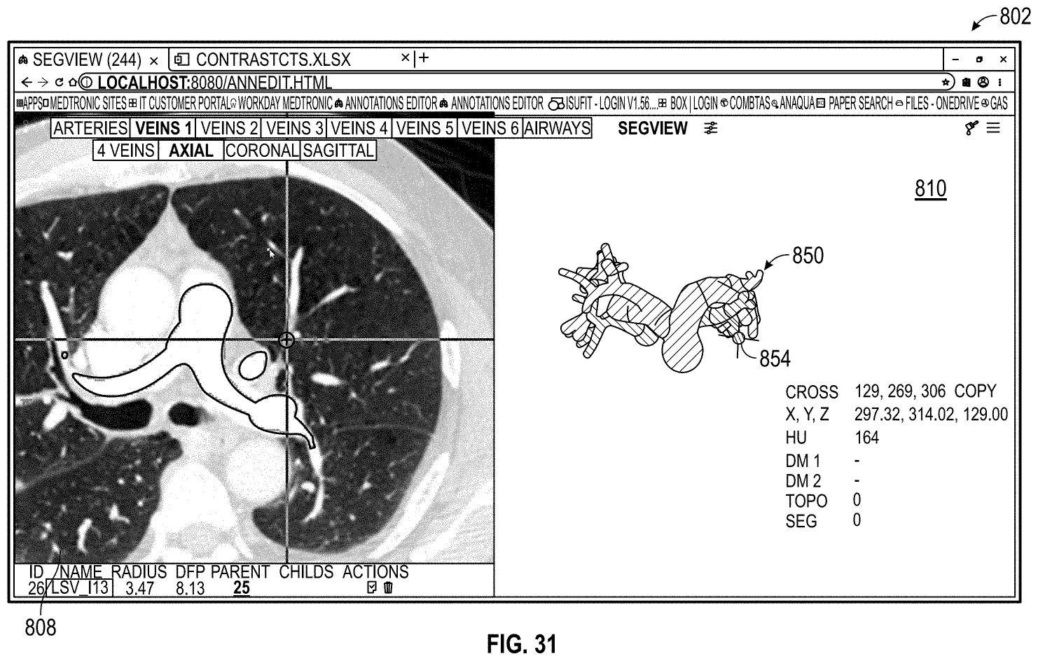

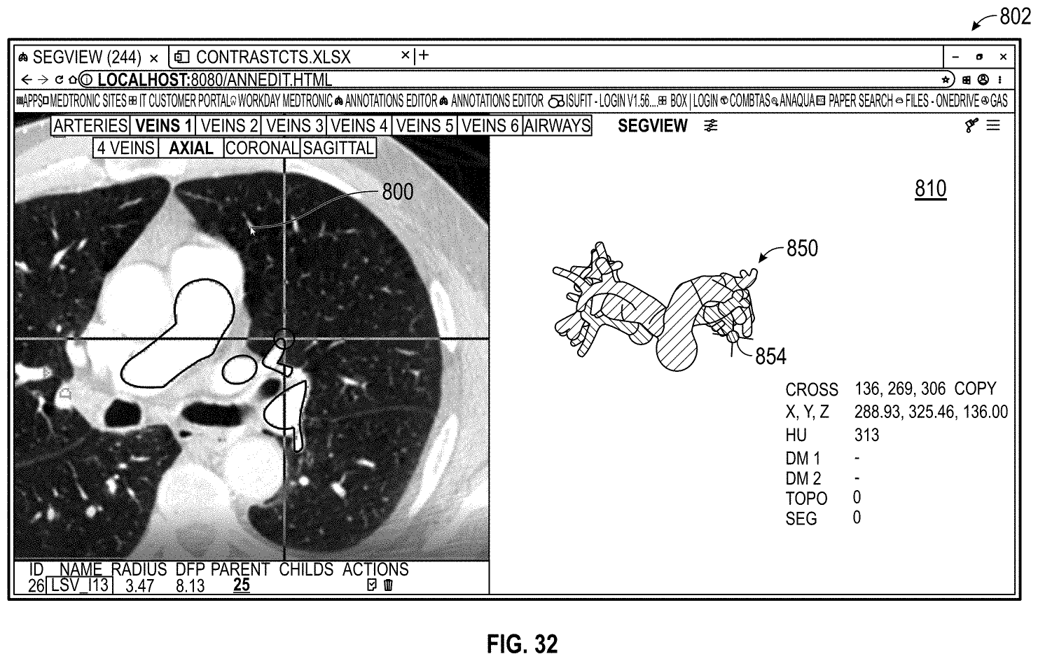

This process, described generally above, can be observed in FIGS. 32-39 and the method is described with reference to both the progressions shown in FIGS. 29-31 and 32-39. As noted above, the image scan data (e.g., CT image data) has been processed, for example via segmentation techniques to assist in distinguishing between different tissue types. As an exemplary starting point for the method of FIG. 27, and as shown in FIG. 30 a 3D model 850 has already been generated in the manner described above. As depicted in FIG. 30, a user may select a tab 852 to individually select one of the coronal 804, sagittal 806, or axial 808 view windows, or present the four views as depicted for example in FIG. 10. In FIG. 30 the axial image window 808 has been selected and is shown on the left side of the UI 802 and the 3D model 850 is shown in the 3D view 810.

The method 1100 starts at step 1102 with the receipt of a user selection of a point 854 in the 3D model 850 (FIG. 30). FIG. 27 is a schematic view 1200 of the segmented image views (e.g., axial image view window 808). The selected point 854 is shown schematically near the end 1202 of a previously generated 3D model 850. At step 1104 the tool 800 determines the closest skeleton point 1204 in the segmented images (i.e., the images displayed in the axial image window 808) to the selected point 854 in the 3D model 850. The determination of the closest skeleton point 1204 also snaps the axial view window 808 (FIG. 30) to the same location as the selected point 854 and displays an indicator 856 around the selected point 854 in the axial view window 808.

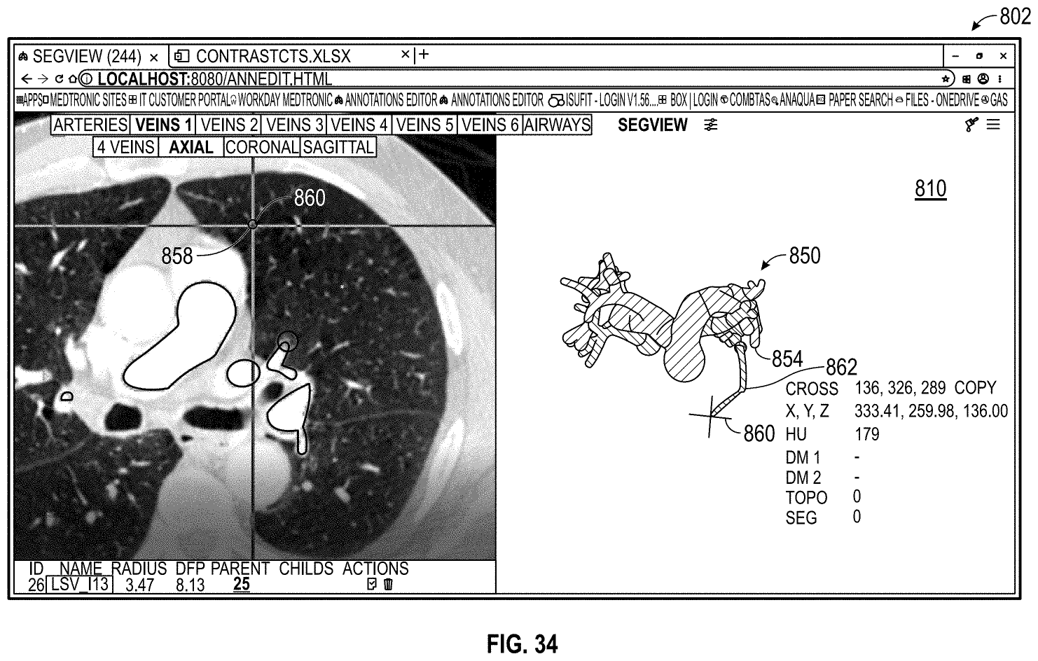

At step 1106 the tool 800 receives a selection of a point (858 FIG. 34) at some location more peripheral in the blood vessel. As can be seen by reviewing the transition from FIG. 30-35, once the skeleton point 1204 is identified and the indicator 856 is depicted in the axial view window 808 the user is free to scroll through the images depicted in the axial view window 808. Because of the segmentation of the image scan data, the white area in which the indicator 856 is placed indicates the blood vessel 860 to be followed and added to the 3D model 850. By scrolling through the images in the axial view window 808 the blood vessel 860 (i.e., the white portion that is connected to the indicator 856) and its advancement from image to image (i.e., its connectedness) can be observed. Though the blood vessel 860 appears to disappear in FIG. 32, in fact, its orientation relative to the axial view is merely different, and careful observation reveals that the blood vessel 860 in the particular image displayed in the axial view window 808 is found closer to the periphery of the lung and has a much different observable dimension in that particular image.

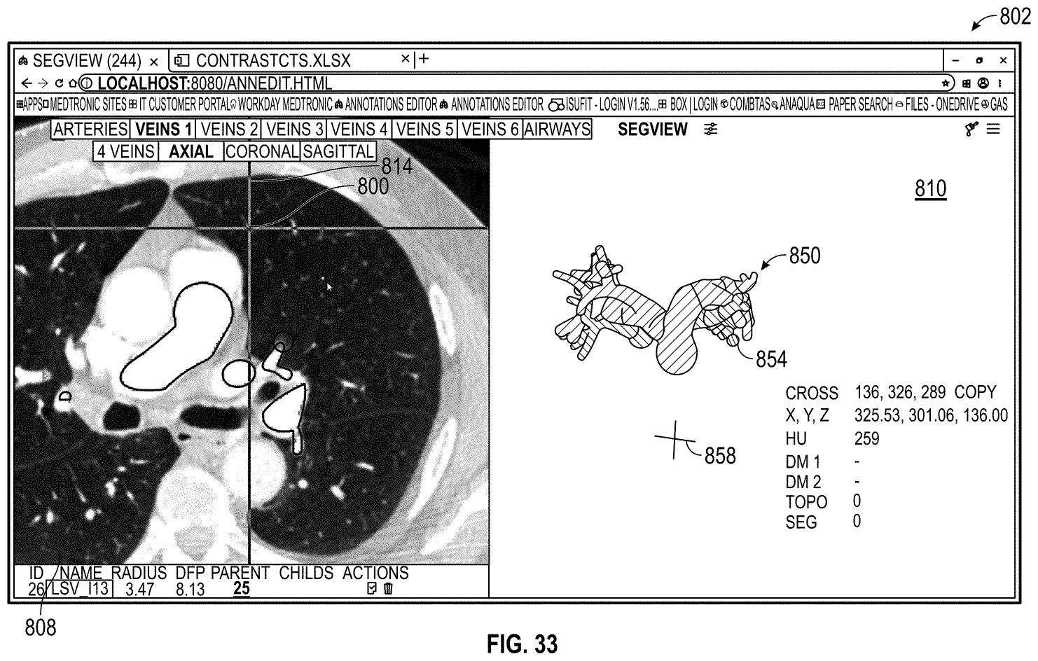

In FIG. 33, the crosshairs 814 are moved to the location at which the blood vessel 860 appears in that image. In FIG. 34 the selection of point 858 is seen at this second point along the blood vessel 860 completing step 1204, described above. This point 858 can also be seen in FIG. 27. As with the selection of the initial point 854, after selection of the point 858, at step 1108 the tool 800, employing method 1100 computes a closest skeleton point 1204 to the point 858. At step 1110 the shortest path 1206 between point 858 and point 854 is calculated. Further at step 1112 the radius 1208 along the length of the path 1206 is calculated. A step 1114 a graph of the shortest path 1206, having the calculated radii is connected to the 3D model.

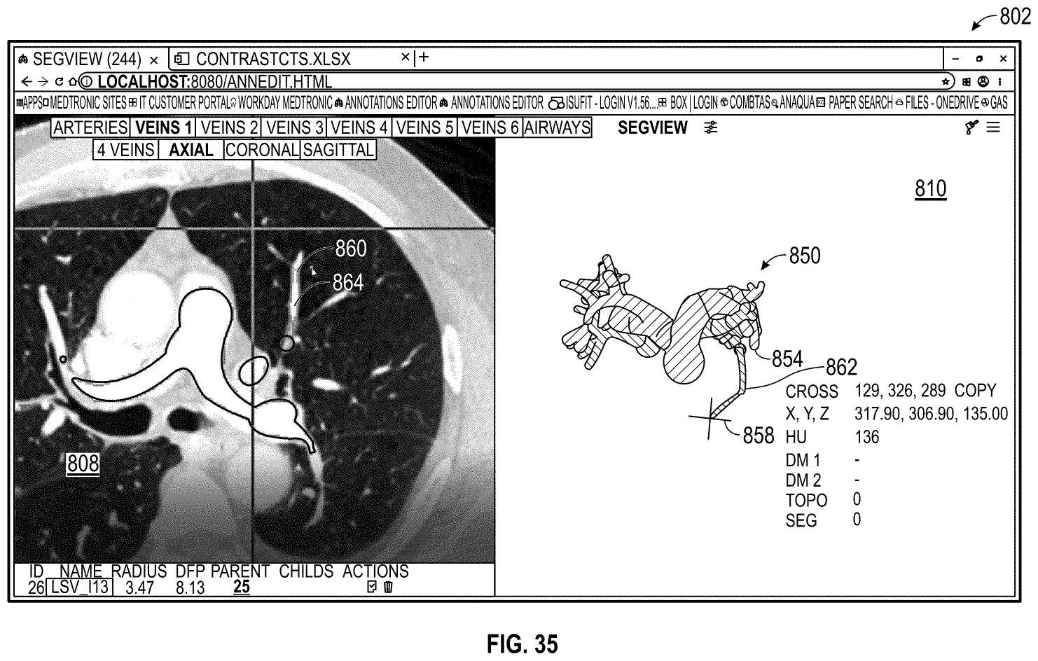

The results can be seen in FIG. 35, where the section 862 of the 3D model 850 extending between points 858 and 854 is generated and displayed on the 3D view 810. Section 862 represents the blood vessel 860 identified in the axial view window 808. In addition, the axial view window 808 also depicts a marker 864 outlining the blood vessel 860 showing the calculated radii and extending back to the indicator 856 of the selected point 854.

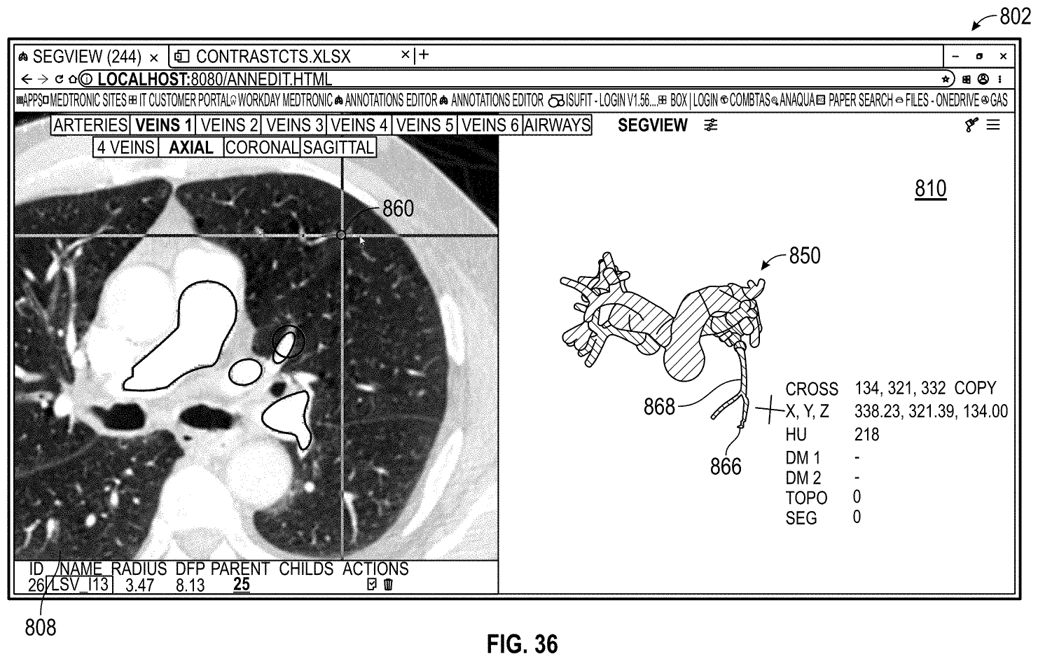

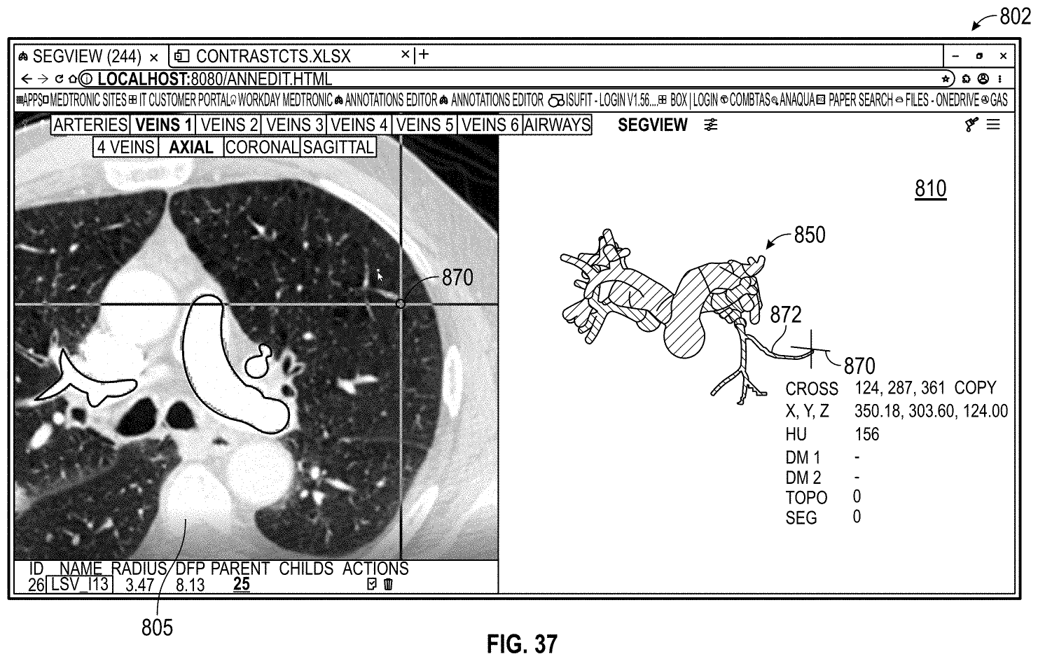

Referring back to method 1100, if at step 1116 it is determined there are no more blood vessels 860 to generate in the 3D model, the method ends, however, if there is a desire to add more blood vessels 860 to the 3D model 850, the method returns to step 1106. As depicted in FIG. 36 a further point 866 is selected peripherally from the indicator 856 but also observed as connected to the blood vessel 860. As depicted in FIG. 30, following steps 1106-1114, the closest segmentation point 1204 to point 866 is found, the shortest path to the first segmentation point 1204 proximate point 854 is determined, the radius of each point along the path is measured and the section 868 representing this portion of the blood vessel 860 is displayed as part of the 3D model 850. Following a similar process selection of a further point 870, depicted in FIG. 37 enables generation of another section 872 of the 3D model.

FIG. 29 depicts, the segments 862 and 868 schematically joined to the 3D model 850 by the processes described above. In this manner, starting from any origin within the 3D model 850 that 3D model can be quickly expanded to the periphery of the lungs. Though described here in connection with blood vessels, the same process may be undertaken for airways in the lungs allowing for quick and accurate segmentation and modeling of the airways and blood vessels. At any point during this process of adding sections to the 3D model 850 the user determines that a section does not accurately reflect what is observed in the axial view window 808, the user may select an undo toggle in the tool 800 and the process may be restarted to correct the issue.

Those of skill in the art will recognize that though the method 1100 is described above in connection with quickly expanding an existing 3D model, the disclosure is not so limited, and instead of receiving the selection of point 854 in the 3D model, the selection may be made in the axial image viewer 808 (or any other viewer) to identify the one point within the blood vessel 860. In this manner, the 3D model may be entirely generated using the method 1100.

IFIG. 29FIG. 31FIG. 32FIG. 33FIG. 34FIG. 35FIG. 26

Any of the above aspects and embodiments of the present disclosure may be combined without departing from the scope of the present disclosure.

While detailed embodiments are disclosed herein, the disclosed embodiments are merely examples of the disclosure, which may be embodied in various forms and aspects. For example, embodiments of an electromagnetic navigation system, which incorporates the target overlay systems and methods, are disclosed herein; however, the target overlay systems and methods may be applied to other navigation or tracking systems or methods known to those skilled in the art. Therefore, specific structural and functional details disclosed herein are not to be interpreted as limiting, but merely as a basis for the claims and as a representative basis for teaching one skilled in the art to variously employ the disclosure in virtually any appropriately detailed structure.

We claim:

1. A system for generating a generating a 3D model of vasculature of a patient comprising:
a memory in communication with a processor and a display, the memory storing instructions that when executed by the processor:
cause a display to display a plurality of images from an image data set in a user interface, the images including at least an axial, sagittal, and coronal view;
receive instructions to scroll through at least one of the axial, sagittal, and coronal images;
receive an indication to create a received indication of a position in one of the axial, sagittal, and coronal images being within a first portion of a vasculature;
snap the remaining images to the position of the received indication;
display crosshairs on the images at the position of the received indication;
depict the position as a first point in a three-dimensional (3D) view;
receive inputs to adjust a level of zoom or a location of the crosshairs in the images;
receive an indication that all three crosshairs are located in the center of the first portion of the vasculature;
depict a second point in a 3D view at the location of all three crosshairs;
depict the first point in an oblique view of the image data set;
depict a circle in the oblique view around the first point;
receive an input to size the size of the circle to match a diameter of the first portion of the vasculature at the second point;
receive an input to add a segment; and
display the segment in the 3D view, wherein the segment extends from the first point to a first node at the location of the second point.

2. The system of claim 1, wherein a depiction of the segment is also presented in the axial, sagittal, and coronal images.

3. The system of claim 1, wherein the segment has a diameter matching the size of the circle around the first point.

4. The system of claim 2, wherein where further segments of the first portion of the vasculature remain unmodeled, the processor executes instructions to:
receive an input to scroll through the images in at least one of the axial, sagittal, and coronal images;
receive an input identifying a third point in the first portion of the vasculature in at least one of the axial, sagittal, and coronal images;
depict a circle in the oblique view around the second point;
receive an input to size the size of the circle to match a diameter of the first portion of the vasculature;
receive an input to add a segment; and
display the segment in the 3D view, wherein the segment extends from the first node to a second node at the location of the third point.

5. The system of claim 4, wherein the instructions are executed in a repeating fashion until all of the first portion of the vasculature is modeled.

6. The system of claim 5, wherein following modeling of all the segments of the first portion of the vasculature, the processor executes instructions to:
receive instructions to scroll through at least one of the axial, sagittal, and coronal images;
receive an indication of a position of one of the axial, sagittal, and coronal images being within a second portion of the vasculature;
snap the remaining images to the position of the received indication;
display crosshairs on the images at the position of the received indication;
depict the position as a first point in the 3D view;
receive inputs to adjust a level of zoom or a location of the crosshairs in the images; and
receive an indication that all three crosshairs are located in the center of the vasculature.

7. The system of claim 6, further comprising:
depict a second point in the 3D view at the location of all three crosshairs;
depict the first point in an oblique view of the image data set;
depict a circle in the oblique view around the first point;
receive an input to size the size of the circle to match a diameter of the second portion of the vasculature;
receive an input to add a segment; and
display the segment in the 3D view, wherein the segment extends from the first point to a first node at the location of the second point.

8. The system of claim 7, wherein the first portion of the vasculature are arteries and the second portion of the vasculature are veins.

9. The system of claim 7, wherein the processor executes instructions to export a 3D model formed of a plurality of the segments to an application for planning a thoracic surgery.

10. The system of claim 7, further comprising identifying an error in at least one segment of a 3D model formed of a plurality of the segments and inserting a segment before the segment with the error.

11. The system of claim 10, wherein following identification of a node is defined between the nodes of the segment containing the error.

12. The system of claim 11, wherein a diameter of the inserted segment is defined in the oblique view.

13. A system for correcting a three-dimensional (3D) model of vasculature of a patient comprising:
a memory in communication with a processor and a display, the memory storing instructions that when executed by the processor:
select a 3D model for presentation on a display;
present the 3D model and axial, coronal and sagittal images from which the 3D model is derived on a user interface;
receive an input to scroll or zoom one or more of the images, or receive a selection of a segment of the 3D model;
receive an indication of a first point in a first segment in the 3D model in need of correction;
depict the point in an oblique view of the images;
depict a circle in the oblique view around the first point;
receive an input to size a size of the circle to match a diameter of the vasculature in the oblique view;
receive an input to add a segment; and
display the added segment in the 3D model, wherein the added segment extends from a point defining a beginning of the first segment to the first point in and corrects an error in the 3D model.

14. The system of claim 13, wherein the processor executes the instructions until all of the 3D model is reviewed and corrected.

15. The system of claim 13, wherein segments of the 3D model depict arterial vasculature in a first color and venous vasculature in a second color.

16. The system of claim 13, wherein the processor further executes an instruction to export the correct 3D model to a thoracic surgery planning application.

17. A method of generating a three-dimensional (3D) model of a vasculature of lungs comprising:
displaying a plurality of images from an image data set in a user interface, the images including at least an axial, sagittal, and coronal view;
receiving instructions to scroll through at least one of the axial, sagittal, and coronal images;
receiving an indication to produce a received indication of a position of one of the axial, sagittal, and coronal images being within a first portion of a vasculature;
displaying crosshairs on the axial, coronal and sagittal images at the position of the received indication;
depicting the position as a first point in a three-dimensional (3D) view;
receiving an input to adjust a level of zoom or a location of the crosshairs in the images;
receiving an indication that all three crosshairs are located in the center of the first portion of the vasculature;
depicting a second point in a 3D view at the location of all three crosshairs;
depicting the first point in an oblique view of the image data set;
depicting a circle in the oblique view around the first point;
receiving an input to size the size of the circle to match a diameter of the first portion of the vasculature around the first point;
receiving an input to add a segment; and
displaying the segment in the 3D view, wherein the segment extends from the first point to a first node at the location of the second point.

18. The method of claim 17, wherein a depiction of the segment is also presented in the axial, sagittal, and coronal images.

19. The method of claim 17, wherein the segment has a diameter matching the size of the circle around the first point.

20. The method of claim 17, wherein where further segments of the first portion of the vasculature remain unmodeled:
receiving an input to scroll through the images in at least one of the axial, sagittal, and coronal images;
receiving an input identifying a third point in the first portion of the vasculature in at least one of the axial, sagittal, and coronal images;
depicting a circle in the oblique view around the second point;
receiving an input to size the size of the circle to match a diameter of the first portion of the vasculature;
receiving an input to add a segment; and
displaying the segment in the 3D view, wherein the segment extends from the first node to a second node at the location of the third point.
